SEQUENCE LISTING (58 sequences)

SEQ ID NO: 1 - protein, 28 aa, Artificial Sequence
  DISULFID From 7 to 23
  AMIDATION 28: Xaa represents Tyr-CONH2
SLRRSSCFGGRMDRIGAQSGLGCNSFRX

SEQ ID NO: 2 - protein, 28 aa, Artificial Sequence
  DISULFID From 7 to 23
  SITE 1: Xaa represents MPA-AEEA-Ser
XLRRSSCFGGRMDRIGAQSGLGCNSFRX

SEQ ID NO: 3 - protein, 28 aa, Artificial Sequence
  DISULFID From 7 to 23
  SITE 1: Xaa represents MPA-AEEA-Ser
XLRRSSCFGGRMDRIGAQSGLGCNSFRX

SEQ ID NO: 4 - protein, 28 aa, Artificial Sequence
  DISULFID From 7 to 23
  SITE 1: Xaa represents MPA-Ser
XLRRSSCFGGRMDRIGAQSGLGCNSFRX

SEQ ID NO: 5 - protein, 29 aa, Artificial Sequence
  DISULFID From 7 to 23
  SITE 29: Xaa represents Lys(AEEA-MPA)-CONH2
SLRRSSCFGGRMDRIGAQSGLGCNSFRYX

SEQ ID NO: 6 - protein, 28 aa, Artificial Sequence
  DISULFID From 7 to 23
  SITE 17: Xaa represents Lys(AEEA-MPA)
SLRRSSCFGGRMDRIGXQSGLGCNSFRX

SEQ ID NO: 7 - protein, 28 aa, Artificial Sequence
  DISULFID From 7 to 23
  SITE 12: Xaa represents Lys(AEEA-MPA)
SLRRSSCFGGRXDRIGAQSGLGCNSFRX

SEQ ID NO: 8 - protein, 32 aa, Artificial Sequence
  DISULFID From 11 to 27
  AMIDATION 32: Xaa represents Tyr-CONH2
TAPRSLRRSSCFGGRMDRIGAQSGLGCNSFRX

SEQ ID NO: 9 - protein, 32 aa, Artificial Sequence
  DISULFID From 11 to 27
  SITE 1: Xaa represents MPA-AEEA-Thr
XAPRSLRRSSCFGGRMDRIGAQSGLGCNSFRX

SEQ ID NO: 10 - protein, 32 aa, Artificial Sequence; identical to SEQ ID NO: 9
  DISULFID From 11 to 27
  SITE 1: Xaa represents MPA-AEEA-Thr

SEQ ID NO: 11 - protein, 28 aa, Artificial Sequence
  DISULFID From 7 to 23
  SITE 1: Xaa represents MPA-AEEA-Ser
XLDDSSCFGGDMDDIGAQSGLGCNSFDX

SEQ ID NO: 12 - protein, 28 aa, Artificial Sequence
  DISULFID From 7 to 23
  AMIDATION 28: Xaa represents Tyr-CONH2
SLDDSSCFGGDMDRIGAQSGLGCNSFDX

SEQ ID NO: 13 - protein, 22 aa, Artificial Sequence
  DISULFID From 1 to 17
  AMIDATION 22: Xaa represents Tyr-CONH2
CFGGRIDRIGAQSGLGCNSFRX

SEQ ID NO: 14 - protein, 22 aa, Artificial Sequence
  DISULFID From 1 to 17
  SITE 1: Xaa represents MPA-AEEA-Cys
XFGGRIDRIGAQSGLGCNSFRX

SEQ ID NO: 15 - protein, 24 aa, Artificial Sequence
  DISULFID From 3 to 19
  AMIDATION 24: Xaa represents Tyr-CONH2
SSCFGGRMDRIGAQSGLGCNSFRX

SEQ ID NO: 16 - protein, 24 aa, Artificial Sequence
  DISULFID From 3 to 19
  SITE 1: Xaa represents MPA-AEEA-Ser
XSCFGGRMDRIGAQSGLGCNSFRX

SEQ ID NO: 17 - protein, 22 aa, Artificial Sequence
  DISULFID From 1 to 17
  AMIDATION 22: Xaa represents Tyr-CONH2
CFGGRMDRIGAQSGLGCNSFRX

SEQ ID NO: 18 - protein, 22 aa, Artificial Sequence
  DISULFID From 1 to 17
  SITE 1: Xaa represents MPA-AEEA-Cys
XFGGRMDRIGAQSGLGCNSFRX

SEQ ID NO: 19 - protein, 28 aa, Artificial Sequence
  DISULFID From 7 to 23
  SITE 8: Xaa represents N-Methyl-Phe
SLRRSSCXGGRMDRIGAQSGLGCNSFRX

SEQ ID NO: 20 - protein, 28 aa, Artificial Sequence
  DISULFID From 7 to 23
  SITE 1: Xaa represents MPA-AEEA-Ser
XLRRSSCXGGRMDRIGAQSGLGCNSFRX

SEQ ID NO: 21 - protein, 32 aa, Artificial Sequence
  DISULFID From 10 to 26
  AMIDATION 32: Xaa represents His-CONH2
SPKMVQGSGCFGRKMDRISSSSGLGCKVLRRX

SEQ ID NO: 22 - protein, 25 aa, Artificial Sequence
  DISULFID From 3 to 19
  AMIDATION 25: Xaa represents His-CONH2
SGCFGRKMDRISSSSGLGCKVLRRX

SEQ ID NO: 23 - protein, 22 aa, Artificial Sequence
  DISULFID From 1 to 17
  AMIDATION 22: Xaa represents Tyr-CONH2
CFGGRIDRIGAQSGLGCNSFRX

SEQ ID NO: 24 - protein, 25 aa, Artificial Sequence
  DISULFID From 3 to 19
  SITE 26: Xaa represents Lys(AEEA-MPA)-CONH2
SGCFGRKMDRISSSSGLGCKVLRRH

SEQ ID NO: 25 - protein, 25 aa, Artificial Sequence
  DISULFID From 3 to 19
  AMIDATION 25: Xaa represents His-CONH2
SGCFGRKIDRISSSSGLGCKVLRRX

SEQ ID NO: 26 - protein, 25 aa, Artificial Sequence
  DISULFID From 3 to 19
  SITE 1: Xaa represents MPA-AEEA-Ser
XGCFGRKIDRISSSSGLGCKVLRRX

SEQ ID NO: 27 - protein, 26 aa, Artificial Sequence
  DISULFID From 3 to 19
  SITE 26: Xaa represents Lys(AEEA-MPA)-CONH2
SGCFGRKIDRISSSSGLGCKVLRRHX

SEQ ID NO: 28 - protein, 23 aa, Artificial Sequence
  DISULFID From 1 to 17
  AMIDATION 23: Xaa represents His-CONH2
CFGRKIDRISSSSGLGCKVLRRX

SEQ ID NO: 29 - protein, 23 aa, Artificial Sequence
  DISULFID From 1 to 17
  SITE 1: Xaa represents MPA-AEEA-Cys
XFGRKIDRISSSSGLGCKVLRRX

SEQ ID NO: 30 - protein, 24 aa, Artificial Sequence
  DISULFID From 1 to 17
  SITE 1: Xaa represents MPA-AEEA-Ser
CFGRKIDRISSSSGLGCKVLRRHX

SEQ ID NO: 31 - protein, 23 aa, Artificial Sequence
  DISULFID From 1 to 17
  AMIDATION 23: Xaa represents His-CONH2
CFGRKMDRISSSSGLGCKVLRRX

SEQ ID NO: 32 - protein, 23 aa, Artificial Sequence
  DISULFID From 1 to 17
  SITE 1: Xaa represents MPA-AEEA-Cys
XFGRKMDRISSSSGLGCKVLRRX

SEQ ID NO: 33 - protein, 24 aa, Artificial Sequence
  DISULFID From 1 to 17
  SITE 24: Xaa represents Lys(AEEA-MPA)-CONH2
CFGRKMDRISSSSGLGCKVLRRHX

SEQ ID NO: 34 - protein, 23 aa, Artificial Sequence
  DISULFID From 1 to 17
  SITE 2: Xaa represents N-alpha-methyl-Phe
CXGRKMDRISSSSGLGCKVLRRX

SEQ ID NO: 35 - protein, 23 aa, Artificial Sequence
  DISULFID From 1 to 17
  SITE 2: Xaa represents N-alpha-methyl-Phe
CXGRKMDRISSSSGLGCKVLRRX

SEQ ID NO: 36 - protein, 24 aa, Artificial Sequence
  DISULFID From 1 to 17
  SITE 2: Xaa represents N-alpha-methyl-Phe
CXGRKMDRISSSSGLGCKVLRRHX

SEQ ID NO: 37 - protein, 32 aa, Artificial Sequence
  DISULFID From 10 to 26
  SITE 11: Xaa represents N-alpha-methyl-Phe
SPKMVQGSGCXGRKMDRISSSSGLGCKVLRRX

SEQ ID NO: 38 - protein, 33 aa, Artificial Sequence
  DISULFID From 10 to 26
  SITE 11: Xaa represents N-alpha-methyl-Phe
SPKMVQGSGCXGRKMDRISSSSGLGCKVLRRHX

SEQ ID NO: 39 - protein, 17 aa, Artificial Sequence
  DISULFID From 1 to 17
  AMIDATION 17: Xaa represents Cys-CONH2
CFGRKIDRISSSSGLGX

SEQ ID NO: 40 - protein, 17 aa, Artificial Sequence
  DISULFID From 1 to 17
  SITE 1: Xaa represents MPA-AEEA-Cys
XFGRKIDRISSSSGLGX

SEQ ID NO: 41 - protein, 18 aa, Artificial Sequence
  DISULFID From 1 to 17
  SITE 1: Xaa represents Lys(AEEA-MPA)-CONH2
CFGRKIDRISSSSGLGCX

SEQ ID NO: 42 - protein, 17 aa, Artificial Sequence
  DISULFID From 1 to 17
  AMIDATION 17: Xaa represents Cys-CONH2
CFGRKMDRISSSSGLGX

SEQ ID NO: 43 - protein, 17 aa, Artificial Sequence
  DISULFID From 1 to 17
  SITE 1: Xaa represents MPA-AEEA-Cys
XFGRKMDRISSSSGLGX

SEQ ID NO: 44 - protein, 18 aa, Artificial Sequence
  DISULFID From 1 to 17
  SITE 18: Xaa represents Lys(AEEA-MPA)-CONH2
CFGRKMDRISSSSGLGCX

SEQ ID NO: 45 - protein, 32 aa, Artificial Sequence
  DISULFID From 10 to 26
  AMIDATION 32: Xaa represents His-CONH2
SPKIVQGSGCFGRKIDRISSSSGLGCKVLRRX

SEQ ID NO: 46 - protein, 32 aa, Artificial Sequence
  DISULFID From 10 to 26
  SITE 1: Xaa represents MPA-AEEA-Ser
XPKIVQGSGCFGRKIDRISSSSGLGCKVLRRX

SEQ ID NO: 47 - protein, 33 aa, Artificial Sequence
  DISULFID From 10 to 26
  SITE 33: Xaa represents Lys(AEEA-MPA)-CONH2
SPKIVQGSGCFGRKIDRISSSSGLGCKVLRRHX

SEQ ID NO: 48 - protein, 32 aa, Artificial Sequence
  DISULFID From 10 to 26
  AMIDATION 32: Xaa represents His-CONH2
SPKMVQGSGCFGRRMDRISSSSGLGCRVLRRX

SEQ ID NO: 49 - protein, 32 aa, Artificial Sequence
  DISULFID From 10 to 26
  SITE 1: Xaa represents MPA-AEEA-Ser
XPKMVQGSGCFGRRMDRISSSSGLGCRVLRRX

SEQ ID NO: 50 - protein, 33 aa, Artificial Sequence
  DISULFID From 10 to 26
  SITE 33: Xaa represents Lys(AEEA-MPA)-CONH2
SPKMVQGSGCFGRRMDRISSSSGLGCRVLRRHX

SEQ ID NO: 51 - protein, 32 aa, Artificial Sequence
  DISULFID From 10 to 26
  AMIDATION 32: Xaa represents His-CONH2
SPKMVQGSGCFGDKMDRISSSSGLGCKVLDDX

SEQ ID NO: 52 - protein, 32 aa, Artificial Sequence
  DISULFID From 10 to 26
  SITE 1: Xaa represents MPA-AEEA-Ser
XPKMVQGSGCFGDKMDRISSSSGLGCKVLDDX

SEQ ID NO: 53 - protein, 33 aa, Artificial Sequence
  DISULFID From 10 to 26
  SITE 33: Xaa represents Lys(AEEA-MPA)-CONH2
SPKMVQGSGCFGDKMDRISSSSGLGCKVLDDHX

SEQ ID NO: 54 - protein, 32 aa, Artificial Sequence
  DISULFID From 10 to 26
  SITE 1: Xaa represents MPA-AEEA-Ser
XPKMVQGSGCFGRKMDRISSSSGLGCKVLRRX

SEQ ID NO: 55 - protein, 33 aa, Artificial Sequence
  DISULFID From 10 to 26
  SITE 33: Xaa represents Lys(AEEA-MPA)-CONH2
SPKMVQGSGCFGRKMDRISSSSGLGCKVLRRHX

SEQ ID NO: 56 - protein, 32 aa, Artificial Sequence
  DISULFID From 10 to 26
  SITE 15: Xaa represents Lys(AEEA-MPA)
SPKMVQGSGCFGRKXDRISSSSGLGCKVLRRX

SEQ ID NO: 57 - protein, 32 aa, Artificial Sequence
  DISULFID From 10 to 26
  SITE 19: Xaa represents Lys(AEEA-MPA)
SPKMVQGSGCFGRKMDRIXSSSGLGCKVLRRX

SEQ ID NO: 58 - protein, 33 aa, Artificial Sequence
  Description of Sequence synthetic peptide
XXXXXXXXXXCXXXXXDRIXXXSXLXCXXXXXX

(19) United States
(12) Patent Application Publication
(10) Pub. No.: US 2005/0176641 A1
(43) Pub. Date: Aug. 11, 2005

(54) Long lasting natriuretic peptide derivatives

(75) Inventors: Peter Bakis, Laval (CA); Dominique P. Bridon, Town of Mont-Royal (CA); Julie Carette, Ste-Catherine (CA); France Leclaire, Deux-Montagnes (CA); Roger Leger, Saint-Lambert (CA); Martin Robitaille, Granby (CA)

Correspondence Address: MORRISON & FOERSTER LLP, 425 MARKET STREET, SAN FRANCISCO, CA, 94105-2482, US

(73) Assignee: CONJUCHEM, INC., Montreal (CA)

(21) Appl. No.: 11/040,810

(22) Filed: Jan. 21, 2005

Related U.S. Application Data
(63) Continuation of application No. 10/471,348, filed on Sep. 8, 2003, now abandoned.
(63) Continuation-in-part of application No. 09/623,548, filed on Sep. 5, 2000, now Pat. No. 6,849,714.
(63) Continuation-in-part of application No. 09/657,276, filed on Sep. 7, 2000, now Pat. No. 6,887,470.
(60) Provisional application No. 60/400,199, filed on Jul. 31, 2002.
(60) Provisional application No. 60/400,413, filed on Jul. 31, 2002.

(51) Int. Cl. A61K 38/17; C07K 14/47

(52) U.S. Cl. 514/12; 530/324

(57) ABSTRACT

This invention relates to long lasting natriuretic peptide (NP) derivatives. The NP derivative has a NP peptide and a reactive entity coupled to the NP peptide. The reactive entity is able to covalently bond with a functionality on a blood component. In particular, this invention relates to NP derivatives having an extended in vivo half-life, and method for the treatment of cardiovascular diseases and disorders such as acute decompensated congestive heart failure (CHF) and chronic CHF.

### FIELD OF THE INVENTION

This invention relates to natriuretic peptide (NP) derivatives. In particular, this invention relates to NP derivatives having an extended in vivo half-life, for the treatment of cardio-vascular diseases and disorders such as acute decompensated congestive heart failure (CHF) and chronic CHF, renal disorders and other diseases and disorders.

### BACKGROUND OF THE INVENTION

The natriuretic peptide family includes four structurally related polypeptide hormones: Atrial Natriuretic Peptide (ANP), Brain Natriuretic Peptide (BNP), C-type Natriuretic Peptide (CNP) and, recently discovered, Dendroaspis Natriuretic Peptide (DNP), (Yandle, 1994; Wilhins et al. 1997; Stein and Levin, 1998).

ANP and BNP mediate natriuresis, diuresis, vasodilatation, antihypertension, renin inhibition, antimitogenesis, and lusitropic properties (increase in the heart's rate relaxation). CNP lacks natriuretic actions but possesses vasodilating and growth inhibiting activity (Chen and Burnett, 2000). Collectively, the natriuretic peptide family counterbalances the effects of the renin-angiotensin-aldosterone system (Espiner 1994, Wilkins et al. 1997, Levin et al. 1998). ANP and BNP have been shown to be physiological antagonists of the effects of angiotensin II (Ang II) on vascular tone, aldosterone secretion, renal-tubule sodium reabsorption, and vascular cell growth (Harris et al. 1987, Itoh et al. 1990, Wilkins et al. 1997, Levin et al. 1998). In addition, secretion of vasopressin (Obana et al. 1985) and endothelin-1 (ET-1) (Saijonmaa et al. 1990) are decreased by ANP.

ANP and BNP do not cross the brain-blood barrier (BBB) but they do reach areas near the central nervous system (i.e. subformical organ and hypothalamus). The actions of NPs in the brain reinforce those in the periphery. Natriuretic peptide receptors are present in areas adjacent to the third ventricle that are not separated from the blood by the BBB, a position that allows binding of circulating ANP as well as locally produced peptide (Langub et al., 1995 in Kelly R. and Struthers A. D., 2001).

Biological effects of natriuretic peptides are mediated through the binding and the activation of cell membrane receptors leading to cyclic GMP production in target cells. These include cGMP-dependent protein kinases (PKG), cGMP-gated ion channels and cGMP-regulated phosphodiesterases (Lincoln & Cornwell 1993, de Bold et al. 1996).

Three subtypes of natriuretic peptide receptors have been described: NPR-A, NPR-B and NPR-C. NPR-A and NPR-B are guanylyl cyclases through which the ligands induce the production of cyclic guanosine monophosphate (cGMP) (for review see Maack 1992, Anand-Srivastava & Trachte 1993). NPR-A is thought to mediate many of the effects of ANP and BNP (Maack 1992, Davidson & Struthers 1994) while CNP acts via NPR-B receptors (Koller et al. 1991, Chen & Burnett 1998). NPR-C is a clearance receptor for all three natriuretic peptides, which may signal through alternative pathways (Anand-Srivastava et al. 1990, Levin 1993).

ANP is a 28 amino acid peptide having a 17-amino acid loop formed by an intramolecular disulphide linkage between two cysteine residues, an amino tail of 6 amino acids and a carboxy tail of 5 amino acids. The structure of ANP, the first member of the family to be identified, was first described in 1984 (Kangawa et al. 1984). The atria exhibit the highest levels of ANP gene expression −1% of the total mRNA codes for ANP. ANP mRNA is also found in the ventricle at 1% of the atrial level. Non-cardiac sites that contain ANP include the brain, anterior lobe of the pituitary gland, the lung, and the kidney (Stein and Levin, 1998).

BNP is a 32 amino acid peptide having a 17-amino acid loop formed by an intramolecular disulphide linkage between two cysteine residues, an amino-terminal tail of 9 amino acids and a carboxy-terminal tail of 6 amino acids. BNP, the second member of the NP family, was first detected in 1988 in extracts of porcine brain as it names suggests (Sudoh et al., 1988). However, it was subsequently shown, similarly to ANP, to be expressed primarily in the ventricular myocardium (Minamino et al., 1988; Hosoda et al., 1991) as well as in the brain and amnion (Stein and Levin, 1998). Like ANP, BNP is released into the circulation when the heart is stretched (Kinnunen et al., 1993). Direct studies of BNP secretion from isolated perfused heart (Ogawa et al., Circ. Res. 1991), and from in-vivo and tissue studies in humans (Mukoyama et al., J. Clin. Invest. 1991), showed that 60-80% of cardiac BNP secretion arises from the ventricle.

ANP is shown to have several therapeutic applications such as for hypertension and pulmonary hypertension (Veale et al.), asthma, renal failure, cardiac failure and radiodiagnostic (Riboghene Inc., Press Release 1998).

BNP is shown to have several therapeutic applications such as for hypertension, asthma and inflammatory-related diseases (Ivax Corp., 2001), hypercholesterolemia (BioNumerik Pharmaceuticals Inc, 2000), emesis (BioNumerik Pharmaceuticals Inc, 1996), erectile dysfunction (Ivax Corp., 1998), renal failure (Abraham et al., 1995), cardiac failure and diagnostic of such (Marcus et al., 1995; Miller et al., 1994), solid tumor treatment (BioNumerik Pharmaceuticals Inc, 1999) and protection of common and serious toxicity with placlitaxel in metastatic breast cancer (Hausheer et al., 1998, BioNumerik Pharmaceuticals Inc, 2001).

One the major problem to overcome for the administration of ANP and BNP is their rapid blood circulation clearance. Human ANP has an in vivo half-life of 1 to 5 min (Woods, 1988; Tonolo et al., 1988; Tang et al., 1984); and human BNP has an in vivo half-life of 12.7 min (Smith et al., 2000). Three independent mechanisms are responsible for the rapid clearance of ANP and BNP: 1) binding to NPR-C with subsequent internalization and lysosomal proteolysis; 2) proteolytic cleavage by endopeptidases such as DPP IV, NEP, APA, APP and ACE; and 3) renal secretion. It has been noted that urodilatin, a natriuretic peptide found to be an amino-terminal extended form of ANP, shows that the sole presence of the four additional residues at the N-terminal renders it much more resistant to enzymatic degradation (Kenny et al. 1993). Nevertheless, urodilatin has only an in vivo half-life of approximately 6 min (Carstens et al., 1998).

Several derivatives, analogs, truncations, elongations or constructs of ANP are proposed and/or patented for improving the efficiency and/or the half-life of the native form of ANP; and the related prior art references are listed herein below.

First, native human ANP is disclosed and claimed in U.S. Pat. No. 5,354,900. Peptides with longer or shorter amino-terminal or carboxy-terminal tails of the native ANP sequence are disclosed in U.S. Pat. No. 4,607,023, U.S. Pat. No. 4,952,561, U.S. Pat. No. 4,496,544 and U.S. Pat. No. 6,013,630. Fragments of the native ANP comprising the carboxy-terminal tail and a part of the loop are disclosed in U.S. Pat. No. 4,673,732. Dimers of ANP are proposed in U.S. Pat. No. 4,656,158 and JP application 62,283,996. Different ANP constructs are proposed in JP application 04,077,499, U.S. Pat. No. 5,248,764 and application WO 02/10195.

ANP sequences with truncation of the amino-terminal tail, the carboxy-terminal tail or the loop, elongation of the tails, addition of alkyl group at one of the tails, amino acid substitutions in the tails or in the loop and/or substitution of the cysteine by another bridging group are proposed in U.S. Pat. No. 4,935,492, U.S. Pat. No. 4,757,048, U.S. Pat. No. 4,618,600, U.S. Pat. No. 4,764,504, U.S. Pat. No. 5,212,286, U.S. Pat. No. 5,258,368, U.S. Pat. No. 5,665,704, U.S. Pat. No. 5,846,932, EP application 0,271,041, EP application 0,341,603, application WO 90/14362, U.S. Pat. No. 5,095,004, U.S. Pat. No. 5,376,635, EP application 0,350,318, EP application 0,269,299, U.S. Pat. No. 5,204,328, U.S. Pat. No. 5,057,603, EP application 0,244,169, U.S. Pat. No. 4,816,443, CA patent 1,267,086, EP application 0,303,243, U.S. Pat. No. 4,861,755, U.S. Pat. No. 5,340,920, JP application 05,286,997, U.S. Pat. No. 4,670,540, and U.S. Pat. No. 5,159,061. Linear peptides having a portion thereof that has some similarities with the loop section of ANP are disclosed in U.S. Pat. No. 5,047,397, U.S. Pat. No. 4,804,650 and U.S. Pat. No. 5,449,662.

Also, several number of derivatives, analogs, truncations, elongations and constructs f BNP are proposed and/or patented for improving the efficiency and/or the half-life of the native form of BNP; and the related prior art references are listed herein below.

Native human BNP, amino and carboxy truncations thereof, and amino elongated sequences thereof are disclosed and claimed is U.S. Pat. No. 5,674,710.

Several groups have proposed different modifications of the native human BNP sequences for preventing it from enzymatic degradation or for increasing its activity. These modifications include one or more of the following modifications: truncation of the amino tail; truncation of the carboxy tail; elongation of the amino tail with the prepro sequence or a fragment thereof; addition of an alkyl group at the amino tail or the carboxy tail; and amino acid substitutions in the tails or in the loop; as disclosed in U.S. Pat. No. 5,114,923, U.S. Pat. No. 5,948,761, U.S. Pat. No. 6,028,055, U.S. Pat. No. 4,904,763, application JP 07,228,598 and application WO 98/45329.

All of the above ANP and BNP sequences have a rapid clearance. There is a need for a long lasting natriuretic peptide having an half-life superior than the native form of ANP and BNP and the modified forms of the ANP and BNP sequences disclosed in the prior art.

### SUMMARY OF THE INVENTION

In accordance with the present invention, there is now provided a NP derivative having an extended in vivo half-life when compared with the ones of native ANP or native BNP. More specifically, the present NP derivative comprises a NP peptide having a reactive entity coupled thereto and capable of reacting with available functionalities on a blood component, either in vivo or ex vivo, to form a stable covalent bond and provide a NP peptide-blood component conjugate. Being conjugated to a blood component, the NP peptide is prevented from undesirable cleavage by endogenous enzymes such as NEP and most likely also prevents binding to the NPR-C receptor which is responsible for a large amount of the blood clearance, thereby extending its in vivo half-life and activity. The covalent bonding formed between the NP derivative and the blood component also substantially prevents renal excretion of the NP peptide until the blood component is degraded, thereby also contributing to extend its in vivo half-life to a period of time closer to the half-life of the blood component which can represent an increase of 1 000 to 10 000 times. The reactive entity may be on the N-terminal or the C-terminal of the NP peptide, or on any other available site along the peptidic chain. Optionally, a lysine residue may be added or substituted at the site of the peptidic chain where the reactive entity is attached.

The NP peptide for derivatization according to the present invention is defined by the following formula, where it should be understood that a peptidic bond links Arg₁₈ and Ile₁₉ and the line between Cys₁₁ and Cys₂₇ represents a direct disulfide bridge: 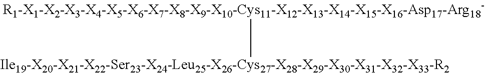
R₁-X₁-X₂-X₃-X₄-X₅-X₆-X₇-X₈-X₉-X₁₀-Cys₁₁-X₁₂-X₁₃-X₁₄-X₁₅-X₁₆-Asp₁₇-Arg₁₈-Ile₁₉-X₂₀-X₂₁-X₂₂-Ser₂₃-X₂₄-Leu₂₅-X₂₆-Cys₂₇-X₂₈-X₂₉-X₃₀-X₃₁-X₃₂-X₃₃-R₂
wherein
X₁ is Thr or absent;
X₂ is Ser, Thr, Ala or absent;
X₃ is Pro, Hpr, Val, or absent;
X₄ is Lys, D-Lys, Arg, D-Arg, Asn, Gln or absent;
X₅ is Met, Leu, Ile, an oxidatively stable Met-replacement amino acid, Ser, Thr or absent;
X₆ is Val, Ile, Leu, Met, Phe, Ala, D-Ala, Nle or absent;
X₇ is Gln, Asn, Arg, D-Arg, Asp, Lys, D-Lys or absent;
X₈ is Gly, Pro, Ala, D-Ala, Arg, D-Arg, Asp, Lys, D-Lys, Gln, Asn or absent;
X₉ is Ser, Thr or absent;
X₁₀ is Gly, Pro, Ala, D-Ala, Ser, Thr or absent;
X₁₂ is Phe, Tyr, Leu, Val, Ile, Ala, D-Ala, Phe with an isosteric replacement of its amide bond selected from the group consisting of Nα-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Arg, Lys, D-Lys, Asp, Gly, Ala, D-Ala or Pro;
X₁₅ is Lys, D-Lys, Arg, D-Arg, Asn, Gln or Asp;
X₁₆ is Met, Leu, Ile or an oxidatively stable Met-replacement amino acid;
X₂₀ is Ser, Gly, Ala, D-Ala or Pro;
X₂₁ is Ser, Gly, Ala, D-Ala, Pro, Val, Leu, or Ile;
X₂₂ is Ser, Gly, Ala, D-Ala, Pro, Gln or Asn;
X₂₄ is Gly, Ala, D-Ala or Pro;
X₂₆ is Gly, Ala, D-Ala or Pro;
X₂₈ is Lys, D-Lys, Arg, D-Arg, Asn, Gln, His or absent;
X₂₉ is Val, Ile, Leu, Met, Phe, Ala, D-Ala, Nle, Ser, Thr or absent;
X₃₀ is Leu, Nle, Ile, Val, Met, Ala, D-Ala, Phe, Tyr or absent;
X₃₁ is Arg, D-Arg, Asp, Lys, D-Lys or absent;
X₃₂ is Arg, D-Arg, Asp, Lys, D-Lys, Tyr, Phe, Trp, Thr, Ser or absent;
X₃₃ is His, Asn, Gln, Lys, D-Lys, Arg, D-Arg or absent;
R₁ is NH₂ or a N-terminal blocking group;
R₂ is COOH, CONH₂ or a C-terminal blocking group.

Preferred blood components comprise proteins such as immunoglobulins, including IgG and IgM, serum albumin, ferritin, steroid binding proteins, transferrin, thyroxin binding protein, α-2-macroglobulin, haptoglobin etc.; serum albumin and IgG being more preferred; and serum albumin being the most preferred.

Reactive entities are capable of forming a covalent bond with the blood component by reacting with amino groups, hydroxy groups, phenol groups or thiol groups present thereon, either in vivo or in vitro. The expressions “in vitro” and “ex vivo” are used in alternance in the specification and means the same in the context of the present invention since what takes place outside the body is performed in vitro. In a preferred embodiment, the functionality on the protein will be a thiol group and the reactive entity will be a Michael acceptor, such as acrolein derivatives, α,β-unsaturated ketones, α,β-unsaturated esters, α,β-unsaturated amides, α,β-unsaturated thioesters, acrylamide, acrylic ester, vinyl benzoate, cinnamate, maleimide or maleimido-containing group such as γ-maleimide-butyrylamide (GMBA) or maleimidopropionic acid (MPA), and the like. The reactive entity can also be iodo methyl benzoate, haloacetates, haloacetamides or the like. MPA is the most preferred reactive entity.

In another aspect of the invention, there is provided a pharmaceutical composition comprising the NP derivative in combination with a pharmaceutically acceptable carrier. Such composition is useful for the treatment of congestive heart failure such as acute decompensated congestive heart failure of NYHA Class II, III and IV and chronic congestive heart failure of NYHA Class III and IV. The composition may also be used for the treatment of one of the following disorders or conditions: renal disorder, hypertension, asthma, hypercholesterolemia, inflammatory-related diseases, erectile dysfunction and for protection for toxicity of anti-cancer drugs. Finally, the present NP derivative may also be used for diagnostic or radiodiagnostic purposes.

In a further aspect of the present invention, there is provided a conjugate comprising the present NP derivative covalently bonded to a blood component. The covalent bond between the NP derivative and the blood component may be performed in vivo or ex vivo.

In an embodiment of the present invention, there is provided a method for the treatment of congestive heart failure such as acute decompensated congestive heart failure of NYHA Class II, III and IV and chronic congestive heart failure of NYHA Class III and IV. The method comprises administering to a subject, preferably a mammal, animal or human, an effective amount of the NP derivative or the conjugate thereof, alone or in combination with a pharmaceutically acceptable carrier.

In others embodiment of the present invention, there is provided a method for the treatment of renal disorder, a method for the treatment of hypertension and a method for the treatment of asthma. These methods comprise administering to a subject, preferably a mammal, animal or human, an effective amount of the NP derivative or the conjugate thereof, alone or in combination with a pharmaceutically acceptable carrier.

In a further embodiment of the present invention, there is provided a method for extending the in vivo half-life of a NP peptide in a subject, the method comprising coupling to the NP peptide a reactive group which is capable of forming a covalent bond with a blood component, and covalently bonding the NP derivative to a blood component. The covalent bonding may take place in vivo or in vitro.

According to the present invention, the NP peptide or fragment thereof possesses natriuretic, diuretic, vasorelaxant and/or renin-angiotensin-aldosterone system modulating activity. Details of the sequences of these peptides and fragments are illustrated below.

In another embodiment of the present invention, the reactive entity is coupled to the NP peptide via a linking group. In this case, the linking group is preferably defined as, without limitation, a straight or branched C₁₋₁₀ alkyl; a straight or branched C₁₋₁₀ alkyl partly or perfluorinated; a C₁₋₁₀ alkyl or fluoroalkyl wherein one or more carbon atom is replaced with O, N or S to form an ether or a thioether, o-, m- or p-disubstituted phenyl wherein the substituents are the same or different and are CH₂, O, S, NH, NR wherein R is H, C₁₋₁₀ alkyl or C₁₋₁₀ acyl; or disubstituted heterocycles such as furan, thiophene, pyran, oxazole, or thiazole. The linking group can be stable or releasable so as to free the NP peptide if desired.

### DESCRIPTION OF THE DRAWINGS

FIG. 1 shows the superposition of the LC/MS profiles of a NP peptide before and after cyclisation performed with the iodine method.

FIG. 2 shows the binding activity of commercial human ANP (hANP), synthesized human ANP (native ANP) and four NP conjugates to guinea pig adrenal gland membranes by displacement of ¹²⁵I-rANP.

FIG. 3 shows the binding activity of synthesized human BNP (native BNP) and four NP conjugates to guinea pig adrenal gland membranes by displacement of ¹²⁵I-rANP.

FIGS. 4 and 5 show the increase of cGMP production in human HELA cells being incubated with in-house synthetized human ANP (native ANP), five NP conjugates and two NP peptides.

FIG. 6 shows the increase of cGMP production in human HELA cells being incubated with in-house synthetized human BNP (native BNP) and four NP conjugates.

FIG. 7 shows in vitro degradation in human plasma of hANP versus two corresponding NP conjugates.

FIG. 8 illustrates the site of cleavage of NEP enzyme along the hANP sequence.

FIG. 9 shows in vitro degradation by NEP enzyme of hANP versus a corresponding NP conjugate, and capped human serum albumin as reference. FIG. 10 shows the pharmacokinetic in rats of hANP (of commercial source and being synthetized in-house) versus two corresponding NP conjugates.

### DESCRIPTION OF THE TABLES

Table 1 shows the three-letter code and one-letter code of amino acids.

Table 2 shows the retention times of NP peptides and NP derivatives according to the present invention.

Tables 3, 4 and 5 show three different gradients of elution of HPLC used for the analysis of NP peptide and NP derivatives of the present invention.

Tables 6 and 7 compare the predicted and measured molecular weight of NP peptides, NP derivatives and NP conjugates.

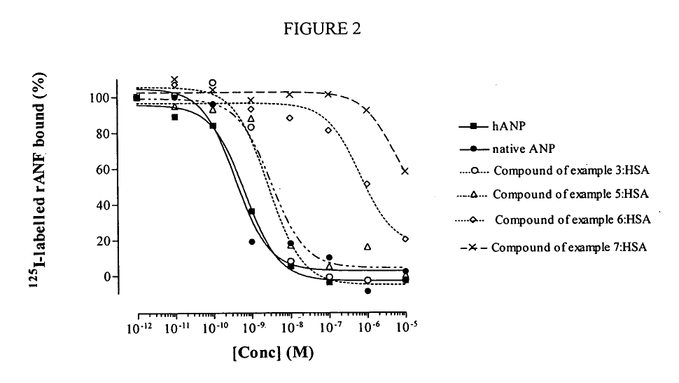

Table 8 shows the concentrations of 50% inhibition (EC50) and the inhibition constants (KI) calculated from the data used to draft FIG. 2 i.e. binding activity of commercial human ANP (hANP), synthesized human ANP (native ANP) and four NP conjugates to guinea pig adrenal gland membranes by displacement of ¹²⁵I-rANP.

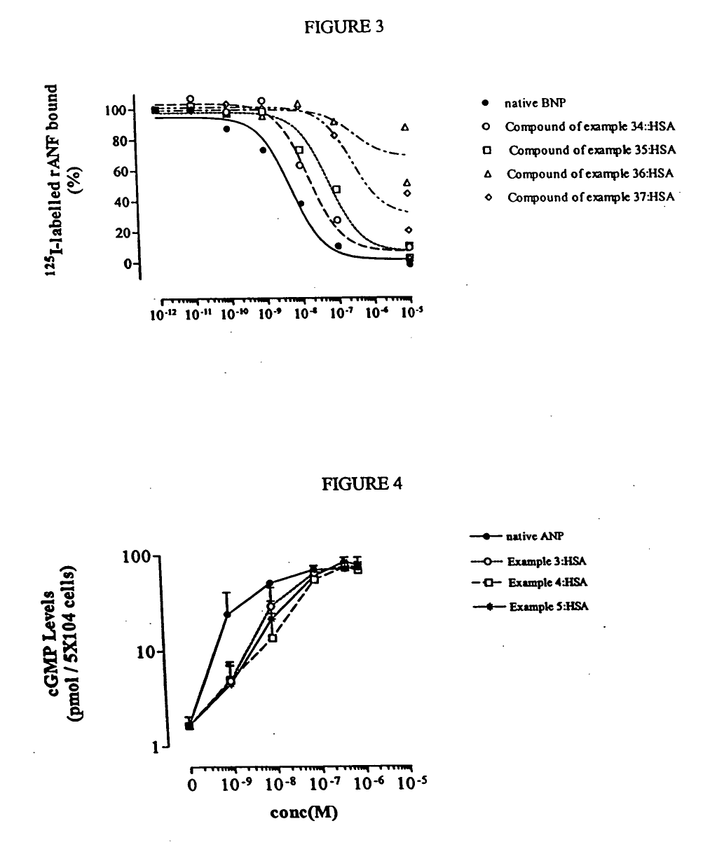

Table 9 shows the concentrations of 50% inhibition (EC50) and the inhibition constants (KI) calculated from the data used to draft FIG. 3 i.e. binding activity of synthetized human BNP (native BNP) and four NP conjugates to guinea pig adrenal gland membranes by displacement of ¹²⁵I-rANP.

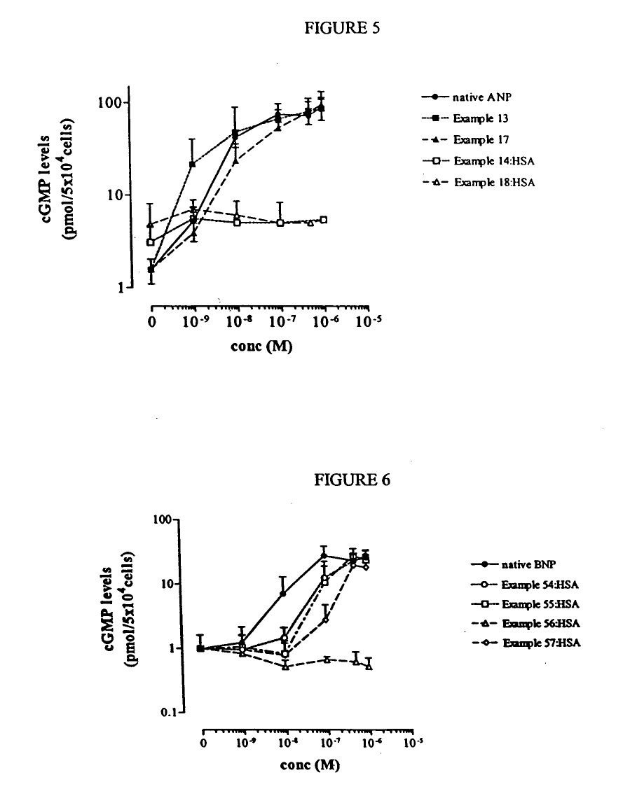

Table 10 lists the concentration of 50% inhibition (EC50) calculated from the data used to draft FIGS. 4, 5 and 6 i.e. the increase of cGMP production in human HELA cells being incubated with in-house synthetized human ANP (native ANP); in-house synthetized human BNP (native BNP); nine NP conjugates; and two NP peptides.

Tables 11 and 12 show the gradients of elution of HPLC respectively used for the analysis of NP peptides and NP derivatives of the present invention.

Tables 13 and 14 show the in vivo effect of the injection of an NP derivative in SHR rats and Winstar-Kyoto rats respectively, on the increase of urine secretion and the increase of cGMP expression.

### DETAILED DESCRIPTION OF THE INVENTION

In vivo bioconjugation is the process of covalently bonding a molecule, such as the NP derivative according to the present invention, within the body, to the targeted blood component, preferably a blood protein, in a manner that permits the substantial retention, or increase in some instances, of the biological activity of the original unmodified NP peptide in the conjugate form, while providing an extended duration of the biological activity though giving the NP peptide the biophysical parameters of the targeted blood component.

According to the invention, the present NP derivative comprise a NP peptide that has been chemically modified by coupling thereto a reactive entity, either directly or via a linking group which is a stable or releasable linking group. The reactive entity is capable of forming a covalent bond with a blood component, preferably a blood protein. The reactive entity must be stable in an aqueous environment. The covalent bond is generally formed between the reactive entity and an amino group, a hydroxyl group, or a thiol group on the blood component. The amino group preferably forms a covalent bond with reactive entities like carboxy, phosphoryl or acyl; the hydroxyl group preferably forms a covalent bond with reactive entities like activated esters; and the thiol group preferably forms a covalent bond with reactive entities like esters or mixed anhydrides. The preferred blood components are mobile blood components like serum albumin, immunoglobulins, or combinations thereof, and the preferred reactive entity comprises anhydrides like maleimide or maleimido-containing groups. In a most preferred embodiment, the blood component is serum albumin and the reactive group is a maleimide-containing group.

Protective groups may be required during the synthesis process (which is described in detail below) to avoid interreaction between the reactive entity and the functional groups of the NP peptide itself. These protective groups are conventional in the field of peptide synthesis, and can be generically described as chemical moieties capable of protecting the peptide derivative from reacting with other functional groups. Various protective groups are available commercially, and examples thereof can be found in U.S. Pat. No. 5,493,007 which is hereby incorporated by reference. Typical examples of suitable protective groups include acetyl, fluorenylmethyloxycarbonyl (FMOC), t-butyloxycarbonyl (BOC), benzyloxycarbonyl (CBZ), etc.

As above-mentioned, conjugation to a blood component definively plays a major role in preventing the NP peptide from degradation by endogenous enzymes such as NEP and preventing binding to the NPR-C receptor which the most important factor for the elimination of the natriuretic peptide from blood circulation. Conjugation to a blood component also overcomes renal excretion of the NP peptide as long as the blood component itself is being degraded. Therefore, the intrinsec half-life of the blood component selected for conjugation is the major determinant for the half-life of the conjugated NP peptide.

The blood components are preferably mobile, which means that they do not have a fixed situs for any extended period of time, generally not exceeding 5 minutes, and more usually one minute. These blood components are not membrane-associated and are present in the blood for extended periods. Preferred mobile blood components include serum albumin, transferrin, ferritin, heptoglobin types 1-1, 2-1, 2-2 and immunoglobulins such as IgM, IgA and IgG.

In greater details, the present invention is directed to the modification of NP peptides and fragments thereof to improve their bioavailability, extend their in vivo half-life and distribution through selective conjugation to a blood component while substantially maintaining or improving their remarkable therapeutic properties.

According to the invention, NP peptide is a peptide having at least one of the physiologic activities of a native ANP or BNP, and particularly of human ANP and BNP. More particularly, NP peptide has natriuretic, diuretic, vasorelaxant and/or renin-angiotensin-aldosterone system modulating activity.

Table 1 provides the three-letter code and one-letter code for natural amino acids and the three-letter code for non-natural amino acids.

TABLE 1
NOMENCLATURE FOR AMINO ACIDS

| Name | 3-letter code | 1-letter code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Norleucine | Nle | |
| Ornithine | Orn | |

The design of the NP peptide for derivatization according to the present invention is based on the sequence of native human ANP and BNP. Their sequences share very high similarities. Substitution by analogous amino acids are proposed for residues that seem less involved in the pharmaceutical activity according to our structural activity analysis. Therefore, the NP peptide according to the present invention corresponds to the sequence of the following formula, where it should be understood that a peptidic bond links Arg₁₈ and Ile₁₉ and the line between Cys₁₁ and Cys₂₇ represents a direct disulfide bridge that forms a loop in the sequence: 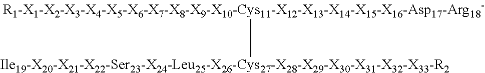
R₁-X₁-X₂-X₃-X₄-X₅-X₆-X₇-X₈-X₉-X₁₀-Cys₁₁-X₁₂-X₁₃-X₁₄-X₁₅-X₁₆-Asp₁₇-Arg₁₈-Ile₁₉-X₂₀-X₂₁-X₂₂-Ser₂₃-X₂₄-Leu₂₅-X₂₆-Cys₂₇-X₂₈-X₂₉-X₃₀-X₃₁-X₃₂-X₃₃-R₂
wherein
X₁ is Thr or absent;
X₂ is Ser, Thr, Ala or absent;
X₃ is Pro, Hpr, Val, or absent;
X₄ is Lys, D-Lys, Arg, D-Arg, Asn, Gln or absent;
X₅ is Met, Leu, Ile, an oxidatively stable Met-replacement amino acid, Ser, Thr or absent;
X₆ is Val, Ile, Leu, Met, Phe, Ala, D-Ala, Nle or absent;
X₇ is Gln, Asn, Arg, D-Arg, Asp, Lys, D-Lys or absent;
X₈ is Gly, Pro, Ala, D-Ala, Arg, D-Arg, Asp, Lys, D-Lys, Gln, Asn or absent;
X₉ is Ser, Thr or absent;
X₁₀ is Gly, Pro, Ala, D-Ala, Ser, Thr or absent;
X₁₂ is Phe, Tyr, Leu, Val, Ile, Ala, D-Ala, Phe with an isosteric replacement of its amide bond selected from the group consisting of Nα-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Arg, Lys, D-Lys, Asp, Gly, Ala, D-Ala or Pro;
X₁₅ is Lys, D-Lys, Arg, D-Arg, Asn, Gln or Asp;
X₁₆ is Met, Leu, Ile or an oxidatively stable Met-replacement amino acid;
X₂₀ is Ser, Gly, Ala, D-Ala or Pro;
X₂₁ is Ser, Gly, Ala, D-Ala, Pro, Val, Leu, or Ile;
X₂₂ is Ser, Gly, Ala, D-Ala, Pro, Gln or Asn;
X₂₄ is Gly, Ala, D-Ala or Pro;
X₂₆ is Gly, Ala, D-Ala or Pro;
X₂₈ is Lys, D-Lys, Arg, D-Arg, Asn, Gin, His or absent;
X₂₉ is Val, Ile, Leu, Met, Phe, Ala, D-Ala, Nle, Ser, Thr or absent;
X₃₀ is Leu, Nle, Ile, Val, Met, Ala, D-Ala, Phe, Tyr or absent;
X₃₁ is Arg, D-Arg, Asp, Lys, D-Lys or absent;
X₃₂ is Arg, D-Arg, Asp, Lys, D-Lys, Tyr, Phe, Trp, Thr, Ser or absent;
X₃₃ is His, Asn, Gln, Lys, D-Lys, Arg, D-Arg or absent;
R₁ is NH₂ or a N-terminal blocking group;
R₂ is COOH, CONH₂ or a C-terminal blocking group.

According to a first preferred embodiment of the invention,
X₁ is Thr or absent;
X₂ is Ala or absent;
X₃ is Pro or absent;
X₄ is Arg or absent;
X₅ is Ser, Thr or absent;
X₆ is Leu, Ile, Nle, Met, Val, Ala, Phe or absent;
X₇ is Arg, D-Arg, Asp, Lys, D-Lys, Gln, Asn or absent;
X₈ is Arg, D-Arg, Asp, Lys, D-Lys, Gln, Asn or absent;
X₉ is Ser, Thr or absent;
X₁₀ is Ser, Thr or absent;
X₁₂ is Phe, Tyr, Leu, Val, Ile, Ala, D-Ala, Phe with an isosteric replacement of its amide bond selected from the group consisting of Nα-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Gly, Ala, D-Ala or Pro;
X₁₅ is Arg, Lys, D-Lys, or Asp;
X₁₆ is Met, Leu, He or an oxidatively stable Met-replacement amino acid;
X₂₀ is Gly, Ala, D-Ala or Pro;
X₂₁ is Ala, D-Ala, Val, Leu, or Ile;
X₂₂ is Gln or Asn;
X₂₄ is Gly, Ala, D-Ala or Pro;
X₂₆ is Gly, Ala, D-Ala or Pro;
X₂₈ is Asn, Gln, His, Lys, D-Lys, Arg, D-Arg or absent;
X₂₉ is Ser, Thr or absent;
X₃₀ is Phe, Tyr, Leu, Val, Ile, Ala or absent;
X₃₁ is Arg, D-Arg, Asp, Lys, D-Lys or absent;
X₃₂ is Tyr, Phe, Trp, Thr, Ser or absent;
X₃₃ is absent;
R₁ is NH₂ or a N-terminal blocking group;
R₂ is COOH, CONH₂ or a C-terminal blocking group

According to the first preferred embodiment of the invention, the following residues are more preferred:
X₁ is Thr or absent;
X₂ is Ala or absent;
X₃ is Pro or absent;
X₄ is Arg or absent;
X₅ is Ser or absent;
X₆ is Leu or absent;
X₇ is Arg, Asp or absent;
X₈ is Arg, Asp or absent;
X₉ is Ser or absent;
X₁₀ is Ser or absent;
X₁₂ is Phe or Phe with an isosteric replacement of its amide bond selected from the group consisting of Nα-methyl, methyl amino, hydroxylethyl, hydrazino, ethylene, sulfonamide and N-alkyl-β-aminopropionic acid;
X₁₃ is Gly;
X₁₄ is Gly,
X₁₅ is Arg or Asp;
X₁₆ is Met or Ile;
X₂₀ is Gly,
X₂₁ is Ala;
X₂₂ is Gln;
X₂₄ is Gly;
X₂₆ is Gly,
X₂₈ is Asn or absent;
X₂₉ is Ser or absent;
X₃₀ is Phe or absent;
X₃₁ is Arg, Asp or absent;
X₃₂ is Tyr or absent;
X₃₃ is absent;
R₁ is NH₂ or a N-terminal blocking group;
R₂ is COOH, CONH₂ or a C-terminal blocking group.

Native human ANP is among the NP peptides in accordance with first embodiment of the present invention. Further preferred NP peptides in accordance with the first embodiment of the present invention are SEQ ID NO: 1, SEQ ID NO: 8, SEQ ID NO: 12, SEQ ID NO: 13, SEQ ID NO: 15, SEQ ID NO: 17 and SEQ ID NO: 19. Preferred NP derivatives, comprising NP peptides according to the first embodiment of the present invention, are SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 10, SEQ ID NO:11, SEQ ID NO: 14, SEQ ID NO: 16, SEQ ID NO:18 and SEQ ID NO: 20.

According to a second preferred embodiment of the invention:
X₁ is absent;
X₂ is Ser, Thr or absent;
X₃ is Pro, Hpr, Val or absent;
X₄ is Lys, D-Lys, Arg, D-Arg, Asn, Gln or absent;
X₅ is Met, Leu, Ile, an oxidatively stable Met-replacement amino acid or absent;
X₆ is Val, Ile, Leu, Met, Phe, Ala, D-Ala, Nle or absent;
X₇ is Gln, Asn or absent;
X₈ is Gly, Pro, Ala, D-Ala or absent;
X₉ is Ser, Thr or absent;
X₁₀ is Gly, Pro, Ala, D-Ala or absent;
X₁₂ is Phe, Tyr, Leu, Val, Ile, Ala, D-Ala, Phe with an isosteric replacement of its amide bond selected from the group consisting of Nα-methyl, methyl amino, hydroxylethyl, hydrazino, ethylene, sulfonamide and N-alkyl->aminopropionic acid, or a Phe-replacement amino acid conferring on said analog resistance to NEP enzyme;
X₁₃ is Gly, Ala, D-Ala or Pro;
X₁₄ is Arg, Lys, D-Lys, or Asp;
X₁₅ is Lys, D-Lys, Arg, D-Arg, Asn or Gln;
X₁₆ is Met, Leu, He or an oxidatively stable Met-replacement amino acid;
X₂₀ is Ser, Gly, Ala, D-Ala or Pro;
X₂₁ is Ser, Gly, Ala, D-Ala or Pro;
X₂₂ is Ser, Gly, Ala, D-Ala or Pro;
X₂₄ is Gly, Ala, D-Ala or Pro;
X₂₆ is Gly, Ala, D-Ala or Pro;
X₂₈ is Lys, D-Lys, Arg, D-Arg, Asn, Gln or absent;
X₂₉ is Val, Ile, Leu, Met, Phe, Ala, D-Ala, Nle or absent;
X₃₀ is Leu, Nle, Be, Val, Met, Ala, D-Ala, Phe or absent;
X₃₁ is Arg, D-Arg, Asp, Lys, D-Lys or absent;
X₃₂ is Arg, D-Arg, Asp, Lys, D-Lys or absent;
X₃₃ is His, Asn, Gln, Lys, D-Lys, Arg, D-Arg or absent;
R₁ is NH₂ or a N-terminal blocking group;
R₂ is COOH, CONH₂ or a C-terminal blocking group.

According to the second preferred embodiment of the invention, the following residues are more preferred:
X₁ is absent;
X₂ is Ser or absent;
X₃ is Pro or absent;
X₄ is Lys or absent;
X₅ is Met, Ile or absent;
X₆ is Val or absent;
X₇ is Gln or absent;
X₈ is Gly or absent;
X₉ is Ser or absent;
X₁₀ is Gly or absent;
X₁₂ is Phe or Phe with an isosteric replacement of its amide bond selected from the group consisting of Nα-methyl, methyl amino, hydroxylethyl, hydrazino, ethylene, sulfonamide and N-alkyl-β-aminopropionic acid;
X₁₃ is Gly;
X₁₄ is Arg or Asp;
X₁₅ is Lys or Arg;
X₁₆ is Met or Ile;
X₂₀ is Ser.
X₂₁ is Ser.
X₂₂ is Ser.
X₂₄ is Gly;
X₂₆ is Gly;
X₂₈ is Lys, Arg or absent;
X₂₉ is Val or absent;
X₃₀ is Leu or absent;
X₃₁ is Arg, Asp or absent;
X₃₂ is Arg, Asp or absent;
X₃₃ is His or absent.

Native human BNP is among the NP peptides in accordance with second embodiment of the present invention. Further preferred NP peptides in accordance with the second embodiment of the present invention are SEQ ID NO: 21, SEQ ID NO: 22, SEQ ID NO: 23, SEQ ID NO: 25, SEQ ID NO: 28, SEQ ID NO: 31, SEQ ID NO: 34, SEQ ID NO: 37, SEQ ID NO: 39, SEQ ID NO: 42, SEQ ID NO: 45, SEQ ID NO: 48 and SEQ ID NO: 51. Preferred NP derivatives, comprising NP peptides according to the second embodiment of the present invention, are SEQ ID NO: 24, SEQ ID NO: 26, SEQ ID NO: 27, SEQ ID NO: 29, SEQ ID NO: 30, SEQ ID NO: 32, SEQ ID NO: 33, SEQ ID NO: 35, SEQ ID NO: 36, SEQ ID NO: 38, SEQ ID NO: 40, SEQ ID NO: 41, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 54, SEQ ID NO: 55, SEQ ID NO: 56 and SEQ ID NO: 57.

The amino acids of the sequences of the NP peptides given in the present application may be D-amino acids or L-amino acids or combinations thereof, unless otherwise specified. L-amino acids are generally preferred.

In a preferred embodiment of the invention, the functionality on the protein will be a thiol group and the reactive entity will be a maleimide or maleimido-containing group such as γ-maleimide-butyrylamide (GMBA) and maleimidopropionic acid (MPA). The reactive entity can be linked to the NP peptide via a stable or releasable linking group. The linking group corresponds is represented by formula V-W where V is a functional group reacting with the NP peptide and W is a chain moiety attached to the reactive entity. V is an ether, a thioether, a secondary or tertiary amine, a secondary or tertiary amide, an ester, a thioester, an imine, an hydrazone, a semicarbazone, an acetal, an hemi-acetal, a ketal, an hemi-ketal, an aminal, an hemi-aminal, an sulfonate, a sulphate, a sulfonamide, a sulfonamidate, a phosphate, a phophoramide, a phosphonate or a phosphonamidate, and preferably a primary amide. W is any alkyl chain C₁₋₁₀, any fluoroalkyl C₁₋₁₀ or any combination of fluorosubstituted alkyl chain C₁₋₁₀, any ether or thioether containing alkyl or fluoroalkyl chains such as -(Z-CH₂CH₂-Z)ₙ—, -(Z-CF₂CH₂-Z)ₙ—, -(Z-CH₂CF₂-Z)ₙ—, where n=1-4 and Z is either O or S, ortho, meta or para disubstituted benzene with structure like —Y—C₆H₄—, —Y—C₆H₄—Y—, where Y is any combination of CH₂, O, S, NH, NR [R=H, alkyl, acyl], disubstituted heterocycles such as furan, thiophene, pyran, oxazole, or thiazole, preferably an alkyl chain C₁₋₆.

The linking group is preferably selected in the group consisting of hydroxyethyl motifs such as (2-amino)ethoxy acetic acid (AEA), ethylenediamine (EDA), 2-[2-(2-amino)ethoxy)]ethoxy acetic acid (AEEA); one or more alkyl chains (C1-C10) motifs such as glycine, 3-aminopropionic acid (APA), 8-aminooctanoic acid (AOA), 4-aminobenzoic acid (APhA). Preferred linking groups are (2-amino)ethoxy acetic acid (AEA), ethylenediamine (EDA), and 2-[2-(2-amino)ethoxy)]ethoxy acetic acid (AEEA). Examples of combinations of linking group and reactive entity include, without limitations, (AEEA-EDA)-MPA; (AEEA-AEEA)-MPA, (AEA-AEEA)-MPA and the like.

It is also contemplated that one or more additional amino acids may be added or substituted to the peptide at the site of coupling the reactive entity, via a linking group or not, prior to performing such coupling on the added or substituted amino acid, in order to facilitate the coupling procedure. The addition or substitution of amino acid(s) may be made at the N-terminal, the C-terminal, or therebetween. It is preferred to substitute an amino acid of the sequence of the NP peptide with Lys, D-Lys, Orn, D-Orn or 2,4-diaminobutanoic acid (DABA) and couple the reactive group on it, optionally via a linking group. To do so, lysine is the most preferred.

Maleimide groups are most selective for sulfhydryl groups on peptides when the pH of the reaction mixture is kept between 6.5 and 7.4. At pH 7.0, the rate of reaction of maleimido groups with sulfhydryls is 1000-fold faster than with amines. When a stable thioether linkage between the maleimido group and the sulfhydryl is formed, it cannot be cleaved under physiological conditions.

The NP derivatives of the invention can provide specific labeling of blood components. The specific labeling, particularly with a maleimide, offers several advantages. Free thiol groups are less abundant in vivo than amino groups, and as a result, maleimide derivatives covalently bond to fewer proteins. For example, in serum albumin, there is only one free thiol group per molecule. Thus, a NP peptide-maleimide-albumin conjugate will tend to comprise a 1:1 molar ratio of peptide: albumin. In addition to albumin, IgG molecules (class II) also have free thiols. Since IgG molecules and serum albumin make up the majority of soluble proteins in the blood, i.e., about 80-85%, they also make up the majority of the free thiol groups available to covalently bond to a NP derivative having a maleimido-containing group.

Further, even among free thiol-containing blood proteins, specific labeling with a maleimide leads to the preferential formation of peptide-maleimide-albumin conjugates, due to the unique characteristics of albumin itself. The single free thiol group of albumin, highly conserved among species, is located at amino acid residue Cys₃₄. It has been demonstrated recently that the Cys₃₄ of albumin has an increased reactivity relative to free thiols on other free thiol-containing proteins and also compared to thiols on low molecular weight molecules. This is due in part to the unusual pK value of 5.5 for the Cys₃₄ of albumin. This is much lower than typical pK values for cysteine residues in general, which are typically about 8-10. Due to this low pK, under normal physiological conditions, Cys₃₄ of albumin is predominantly in the anionic form, which dramatically increases its reactivity. In addition to the low pK value of Cys₃₄, another factor which enhances the reactivity of Cys₃₄ is its location, which is in a hydrophobic pocket close to the surface of one loop of region V of albumin. This location makes Cys₃₄ accessible to ligands of all kinds, and is an important factor in Cys₃₄'s biological role as free radical trap and free thiol scavenger. As a result, the reaction rate acceleration can be as much as 1000-fold relative to rates of reaction of peptide-maleimides with other free-thiol containing proteins and with free thiols containing low molecular weight molecules.

Another advantage of peptide-maleimide-albumin conjugates is the reproducibility associated with the 1:1 loading of peptide to albumin specifically at Cys₃₄. Conventional activation techniques, such as with glutaraldehyde, DCC, EDC and other chemical activators of, for example, free amines, lack this selectivity. For example, human albumin contains 59 lysine residues, 25-30 of which are located on the surface of albumin and accessible for conjugation. Activating these lysine residues, or alternatively modifying a peptide to couple through these lysine residues, results in a heterogeneous population of conjugates. Even if an equimolar ratio peptide:albumin (i.e., 1:1) is employed, the end result is the production of random conjugation products, some containing an indefinite number of peptides linked to each molecule of albumin, and each conjugate having peptides randomly coupled at any one of the 25-30 available lysine sites. Consequently, characterization of the exact composition is virtually impossible, not to mention the absence of reproducibility. Additionally, while it would seem that conjugation through lysine residues of albumin would at least have the advantage of delivering more therapeutic agent per albumin molecule, studies have shown that a 1:1 ratio of therapeutic agent to albumin is preferred. In an article by Stehle, et al. in Anti-Cancer Drugs, 1997, 8, 677-685, which is incorporated herein in its entirety, it is reported that a 1:1 ratio of the anti-cancer methotrexate to albumin conjugated via glutaraldehyde gave the most promising results. These conjugates were taken up by tumor cells, whereas conjugates bearing 5:1 to 20:1 methotrexate molecules had altered HPLC profiles and were quickly taken up by the liver in vivo. It would is therefore seems that at higher ratios, the effectiveness of albumin as a carrier for a therapeutic agent is diminished.

Through controlled administration of the present NP derivative, and particularly the ones with a maleimide reactive entity, specific in vivo labeling or bonding of albumin and IgG can be controlled. In typical intravenous administrations, it has been shown that 80-90% of the administered peptide derivative bonds to albumin and less than 5% bonds to IgG. Trace bonding of free thiols present, such as glutathione and cysteine, also occurs. Such specific bonding is preferred for in vivo use as it permits an accurate calculation of the estimated half-life of the NP peptide administered. The present invention also relates to NP derivatives being capable of selectively covalently bonding with one functionality on a targeted blood component whith a degree of selectivity of 80% or more. Preferably, the degree of selectivity is 90% or more, and more preferably, 95% or more.

As stated above, the desired conjugates of NP derivatives to blood components may be prepared in vivo by administration of the derivatives directly to the subject, which may be an animal or a human. The administration may be done in the form of a bolus, or introduced slowly over time by infusion using metered flow or the like.

Alternately, the conjugate may also be prepared ex vivo or in vitro by combining blood samples or purified blood components with the NP derivatives, allowing covalent bonding of the NP derivatives to the functionalities on blood components, and the resulting blood solution or the resulting purified blood component conjugates may be administered to the subject, animal or human. The purified blood components can be of commercial source, prepared by recombinant techniques or purified from blood samples. The blood may be treated to prevent coagulation during handling ex vivo.

The invention is also directed to the therapeutic uses and other related uses of NP derivatives and fragments thereof having an extended half-life in vivo, and one or more of the following ANP-associated properties and BNP-associated properties:
hypertension reduction;
diuresis inducement;
natriuresis inducement;
vascular conduct dilatation or relaxation;
natriuretic peptide receptors (such as NPR-A) binding;
liberation suppression of norepinephrine through suppression of sympatic nerve;
renin secretion suppression from kidney,
aldostrerone secretion suppresion from adrenal gland;
treatment of cardiovascular disease and disorder,
reducing, stopping or reversing cardiac remodling process in congestive heart failure;
treatment of renal disease and disorder, and
treatment asthma.

According to the present invention, the NP derivatives or NP conjugates can be administered to patients that would benefit from inducing natriuresis, diuresis and vasodilatation. The NP derivatives and conjugates of the present invention are particularly useful to treat cardiac failure such as congestive heart failure (CHF) and more particularly acute decompensated CHF of NYHA Class II, III and IV and chronic CHF of NYHA Class III and IV. NP derivatives or NP conjugates can be administered in a single dose in acute CHF or following a long term medication for chronic CHF. Also, NP derivatives or NP conjugates can be administered alone or in combination with one or more of the following types of compounds: ACE inhibitors, beta blockers, diuretics, spironolactone, digoxin, anticoagulation and antiplatelet agents, and angiotensin receptor blockers.

Other diseases or conditions can be treated with the administration of NP derivatives and NP conjugates of the present invention and include renal disorders and diseases, asthma, hypertension and pulmonary hypertension. More particularly for the NP derivatives and conjugates based on formula II, the following diseases and conditions can also be treated: inflammatory-related diseases, erectile dysfunction and hypercholesterolemia; and also be used as protectant for toxicity of anti-cancer drugs.

Two or more NP derivatives or conjugates of the present invention may be used in combination to optimize their therapeutic effects. They can be administered in a physiologically acceptable medium, e.g. deionized water, phosphate buffered saline (PBS), saline, aqueous ethanol or other alcohol, plasma, proteinaceous solutions, mannitol, aqueous glucose, alcohol, vegetable oil, or the like. Other additives which may be included include buffers, where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. The compositions may be lyophilized for convenient storage and transport.

The NP derivatives and conjugates of the present invention may be administered orally, pulmonary, parenterally, such as intravascularly (IV), intraarterially (IA), intramuscularly (IM), subcutaneously (SC), or the like. Administration by transfusion may be appropriate in some situations. In some cases, administration may be oral, nasal, rectal, transdermal or by aerosol. It can be suitable to employ a single dose or multiple daily doses so as to build the desired systemic dosage. In the case of chronic use, the inverval of administration are established in relation with subject's needs. The NP derivative or conjugate may be administered by any convenient means, including syringe, trocar, catheter, or the like. The particular manner of administration will vary depending upon the amount to be administered, whether a single bolus or continuous administration, or the like.

The blood of the mammalian host may be monitored for the activity of NP peptides and/or presence of the NP derivatives or conjugates. By taking a blood sample from the host at different times, one may determine whether the NP peptide has become bonded to the long-lived blood components in sufficient amount to be therapeutically active and, thereafter, determine the level of NP peptide in the blood. If desired, one may also determine to which of the blood components the NP peptide is covalently bonded. Monitoring may also take place by using assays of peptide activity, HPLC-MS, antibodies directed to peptides, or fluorescent-labeled or radiolabeled derivatives.

Another aspect of this invention relates to methods for determining the concentration of the NP peptide or its conjugate in biological samples (such as blood) using antibodies specific to the NP peptide and to the use of such antibodies as a treatment for toxicity potentially associated with such NP peptide or conjugate. This is advantageous because the increased stability and life of the NP peptide in the patient might lead to novel problems during treatment, including increased possibility for toxicity. The use of anti-NP antibodies, either monoclonal or polyclonal, having specificity for NP, can assist in mediating any such problem. The antibody may be generated or derived from a host immunized with the particular NP derivative, or with an immunogenic fragment of the NP peptide, or a synthesized immunogen corresponding to an antigenic determinant of the NP peptide. Preferred antibodies will have high specificity and affinity any of the NP peptide, the derivatized form thereof and the conjugated form thereof. Such antibodies can also be labeled with enzymes, fluorochromes, or radiolabels.

Antibodies specific for a particular NP derivative may be produced by using purified NP peptides for the induction of derivatized NP-specific antibodies. By induction of antibodies, it is intended not only the stimulation of an immune response by injection into animals, but analogous steps in the production of synthetic antibodies or other specific binding molecules such as screening of recombinant immunoglobulin libraries. Both monoclonal and polyclonal antibodies can be produced by procedures well known in the art.

The antibodies may also be used to monitor the presence of the NP peptide in the blood stream. Blood and/or serum samples may be analyzed by SDS-PAGE and western blotting. Such techniques allow determination of the level of conjugation of the NP derivative.

The anti-NP antibodies may also be used to treat toxicity induced by administration of the NP derivative, and may be used ex vivo or in vivo. Ex vivo methods would include immunuo-dialysis treatment for toxicity employing anti-therapeutic agent antibodies fixed to solid supports. In vivo methods include administration of anti-NP antibodies in amounts effective to induce clearance of antibody-agent complexes.

The antibodies may be used to remove the NP derivatives and conjugates thereof, from a patient's blood ex vivo by contacting the blood with the antibodies under sterile conditions. For example, the antibodies can be fixed or otherwise immobilized on a column matrix and the patient's blood can be removed from the patient and passed over the matrix. The NP derivatives will bind to the antibodies and the blood containing a low concentration of NP, then may be returned to the patient's circulatory system. The amount of NP derivative removed can be controlled by adjusting the pressure and flow rate. Preferential removal of the NP derivative from the serum component of a patients blood can be effected, for example, by the use of a semipermeable membrane, or by otherwise first separating the serum component from the cellular component by ways known in the art prior to passing the serum component over a matrix containing the anti-therapeutic antibodies. Alternatively the preferential removal of NP-conjugated blood cells, including red blood cells, can be effected by collecting and concentrating the blood cells in the patient's blood and contacting those cells with fixed anti-NP antibodies to the exclusion of the serum component of the patient's blood.

The anti-NP antibodies can be administered in vivo, parenterally, to a patient that has received the NP derivative or conjugates for treatment. The antibodies will bind the NP derivative and conjugates. Once bound, the NP activity will be hindered if not completely blocked thereby reducing the biologically effective concentration of NP derivatives in the patient's bloodstream and minimizing harmful side effects if any. In addition, the bound antibody-NP complex will facilitate clearance of the NP derivative and conjugates from the patient's blood stream.

Direct Attachment of the Reactive Entity

The reactive entity (via a linking group or not), such as MPA, is activated as a succinate ester for example (one skilled in the art can use haloacyl or p-nitrophenyl or others) and reacted with an amino group of NP peptide or derivative thereof produced by Solid Phase Synthesis or by recombinants means (see Example 2). In order to perform such direct attachment of the reactive entity, the amino group is selected from the group consisting of the amino group of the C-terminal residue, the amino group of the N-terminal residue, or the amino group of the lateral chain of an amino acid such as Lys, D-Lys, Orn, D-Orn and DABA.

Peptide Derivative Synthesis

NP peptides may be synthesized by standard methods of solid phase peptide chemistry well known to any one of ordinary skill in the art. For example, the peptide may be synthesized by solid phase chemistry techniques following the procedures described by Steward et al. in Solid Phase Peptide Synthesis, 2nd Ed., Pierce Chemical Company, Rockford, Ill., (1984) using a Rainin PTI Symphony™ synthesizer. Similarly, peptides fragments may be synthesized and subsequently combined or linked together to form a larger peptide (segment condensation). These synthetic peptide fragments can also be made with amino acid substitutions and/or deletion at specific locations.

For solid phase peptide synthesis, a summary of the many techniques may be found in Stewart et al. in “Solid Phase Peptide Synthesis”, W. H. Freeman Co. (San Francisco), 1963 and Meienhofer, Hormonal Proteins and Peptides, 1973, 2 46. For classical solution synthesis, see for example Schroder et al. in “The Peptides”, volume 1, Acacemic Press (New York). In general, such method comprises the sequential addition of one or more amino acids or suitably protected amino acids to a growing peptide chain on a polymer. Normally, either the amino or carboxyl group of the first amino acid is protected by a suitable protecting group. The protected and/or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

After all the desired amino acids have been linked in the proper sequence, any remaining protecting groups (and any solid support) are cleaved sequentially or concurrently to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide (segment condensation).

The particularly preferred method of preparing the present NP derivatives of the present invention is solid phase peptide synthesis where the amino acid α-N-terminal is protected by an acid or base sensitive group. Such protecting groups should have the properties of being stable to the conditions of peptide linkage formation while being readily removable without destruction of the growing peptide chain or racemization of any of the chiral centers contained therein. Examples of N-protecting groups and carboxy-protecting groups are disclosed in Greene, “Protective Groups In Organic Synthesis,” (John Wiley & Sons, New York pp. 152-186 (1981)), which is hereby incorporated by reference. Examples of N-protecting groups comprise, without limitation, loweralkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl, o-nitrophenylsulfonyl, 2,2,5,7,8-pentamethylchroman-6-sulfonyl (pmc), and the like; carbamate forming groups such as t-amyloxycarbonyl, benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (boc), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, isobornyloxycarbonyl, cyclopentyloxycarbonyl, adamantyloxycarbonyl, cyclohexyloxycarbonyl, phenylthiocabonyl and the like; arylalkyl groups such as benzyl, biphenylisopropyloxycarbonyl, triphenylmethyl, benzyloxymethyl, 9-fluorenylmethyloxycarbonyl (Fmoc) and the like and silyl groups such as trimethylsilyl and the like. Preferred α-N-protecting group are o-nitrophenylsulfenyl; 9-fluorenylmethyloxycarbonyl; t-butyloxycarbonyl (boc), isobornyloxycarbonyl; 3,5-dimethoxybenzyloxycarbonyl; t-amyloxycarbonyl; 2-cyano-t-butyloxycarbonyl, and the like, 9-fluorenyl-methyloxycarbonyl (Fmoc) being more preferred, while preferred side chain N-protecting groups comprise 2,2,5,7,8-pentamethylchroman-6-sulfonyl (pmc), nitro, p-toluenesulfonyl, 4-methoxybenzene-sulfonyl, Cbz, Boc, and adamantyloxycarbonyl for side chain amino groups like lysine and arginine; benzyl, o-bromobenzyloxycarbonyl, 2,6-dichlorobenzyl, isopropyl, t-butyl (t-Bu), cyclohexyl, cyclopenyl and acetyl (Ac) for tyrosine; t-butyl, benzyl and tetrahydropyranyl for serine; trityl, benzyl, Cbz, p-toluenesulfonyl and 2,4-dinitrophenyl for histidine; formyl for tryptophan; benzyl and t-butyl for aspartic acid and glutamic acid; and triphenylmethyl (trityl) for cysteine.

A carboxy-protecting group conventionally refers to a carboxylic acid protecting ester or amide group. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups comprise, without limitation, C₁-C₈ loweralkyl; arylalkyl such as phenethyl or benzyl and substituted derivatives thereof such as alkoxybenzyl or nitrobenzyl groups; arylalkenyl such as phenylethenyl; aryl and substituted derivatives thereof such as 5-indanyl; dialkylaminoalkyl such as dimethylaminoethyl; alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1 (propionyloxy)-1-ethyl, pivaloyloxymethyl, propionyloxymethyl; cycloalkanoyloxyalkyl groups such as cyclopropylcarbonyloxymethyl, cyclobutylcarbonyloxymethyl, cyclopentylcarbonyloxymethyl, cyclohexylcarbonyloxy-methyl; aroyloxyalkyl such as benzoyloxymethyl, benzoyloxyethyl; arylalkylcarbonyloxyalkyl such as benzylcarbonyloxymethyl, 2-benzylcarbonyloxyethyl; alkoxycarbonylalkyl or cycloalkyloxycarbonylalkyl such as methoxycarbonylmethyl, cyclohexyloxycarbonylmethyl, 1-methoxycarbonyl-1-ethyl; alkoxycarbonyloxyalkyl or cycloalkyloxycarbonyloxyalkyl such as methoxycarbonyloxymethyl, t-butyloxycarbonyl-oxymethyl, 1-ethoxycarbonyloxy-1-ethyl, 1-cyclohexyloxycarbonyloxy-1-ethyl; aryloxy-carbonyloxyalkyl such as 2-(phenoxycarbonyloxy)ethyl, 25-indanyloxycarbonyloxy)ethyl; alkoxyalkylcarbonyloxyalkyl such as 21-methoxy-2-methylpropan-2-oyloxy)-ethyl; arylalkyloxycarbonyloxyalkyl such as 2-(benzyloxycarbonyloxy)ethyl; arylalkenyloxycarbonyloxyalkyl such as 23-phenylpropen-2-yloxycarbonyloxy)ethyl; alkoxycarbonylaminoalkyl such as t-butyloxycarbonylaminomethyl; alkylaminocarbonyl-aminoalkyl such as methylaminocarbonylaminomethyl; alkanoylaminoalkyl such as acetylaminomethyl; heterocycliccarbonyloxyalkyl such as 4-methylpiperazinyl-carbonyloxymethyl; dialkylaminocarbonylalkyl such as dimethylaminocarbonylmethyl, diethylaminocarbonylmethyl; (5-(loweralkyl)-2-oxo-1,3-dioxolen-4-yl)alkyl such as (5-t-butyl-2-oxo-1,3-dioxolen-4-yl)methyl; and (5-phenyl-2-oxo-1,3-dioxolen-4-yl)alkyl such as (5-phenyl-2-oxo-1,3-dioxolen-4-yl)methyl. Representative amide carboxy protecting groups comprise, without limitation, aminocarbonyl and loweralkylaminocarbonyl groups. Of the above carboxy-protecting groups, loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester are preferred. Preferred amide carboxy protecting groups are loweralkylaminocarbonyl groups.

In the solid phase peptide synthesis method, the α-C-terminal amino acid is attached to a suitable solid support or resin. Suitable solid supports useful for the above synthesis are those materials that are inert to the reagents and reaction conditions of the stepwise condensation-deprotection reactions, as well as being insoluble in the media used. The preferred solid support for synthesis of α-C-terminal carboxy peptides is a Ramage Amide Linker™ Resin (R. Ramage et al., THL, 34, p. 6599 (1993)). The preferred solid support for α-C-terminal amide peptides Fmoc-protected Ramage Amide Linker™ Resin.

When the solid support is 42′,4′-dimethoxyphenyl-Fmoc-aminomethyl)-phenoxy-acetamidoethyl resin, the Fmoc group is cleaved with a secondary amine, preferably piperidine, prior to coupling with the α-C-terminal amino acid as described above. The preferred method for coupling to the deprotected 4-(2′,4′-dimethoxyphenyl-Fmoc-aminomethyl)phenoxy-acetamidoethyl resin is O-benzotriazol-1-yl-N,N,N′,N′-tetramethyluroniumhexafluoro-phosphate (HBTU, 5 equiv.), diisopropylethylamine (DIEA, 5 equiv.), and optionally 1-hydroxybenzotriazole (HOBT, 5 equiv.), in DMF. The coupling of successive protected amino acids can be carried out in an automatic polypeptide synthesizer in a conventional manner as is well known in the art.

The removal of the Fmoc protecting group from the α-N-terminal side of the growing peptide is accomplished conventionally, for example, by treatment with a secondary amine, preferably piperidine. Each protected amino acid is then introduced in about 6-fold molar excess, and the coupling is preferably carried out in DMF. The coupling agent is normally O-benzotriazol-1-yl-N,N,N′,N′-tetramethyluroniumhexafluoro-phosphate (HBTU, 5 equiv.), diisopropylethylamine (DIEA, 5 equiv.), and optionally 1-hydroxybenzotriazole (HOBT, 5 equiv.).

At the end of the solid phase synthesis, the peptide is removed from the resin and deprotected, either in successive operations or in a single operation. Removal of the polypeptide and deprotection can be accomplished conventionally in a single operation by treating the resin-bound polypeptide with a cleavage reagent comprising thioanisole, triisopropylsilane, phenol, and trifluoroacetic acid. In cases wherein the α-C-terminal of the polypeptide is an alkylamide, the resin is cleaved by aminolysis with an alkylamine. Alternatively, the peptide may be removed by transesterification, e.g. with methanol, followed by aminolysis or by direct transamidation. The protected peptide may be purified at this point or taken to the next step directly. The removal of the side chain protecting groups is accomplished using the cleavage mixture described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (such as Amberlite XAD™); silica gel adsorption chromatography; ion exchange chromatography on carboxymethylcellulose; partition chromatography, e.g. on Sephadex G-25™, LH-20™ or countercurrent distribution; high performance liquid chromatography (HPLC), especially reverse-phase HPLC on octyl- or octadecylsilyl-silica bonded phase column packing. Anyone of ordinary skill in the art will be able to determine easily what would be the preferred chromatographic steps or sequences required to obtain acceptable purification of the NP peptide.

NP peptides and derivatives are cyclic. For the cyclisation, the thiol groups of the peptide can be reduced by a tallium, iodine or by the sulphoxide method. The iodine method is exemplified herein below in Example 1 and the sulphoxide method is exemplified herein below in Examples 3, 5, 21 and 24. When the peptide has a reactive entity, and more particularly when the reactive entity is MPA, the cyclisation is preferably made with the sulphoxide method.

After the cyclisation step, a final purification is performed on the cyclised product. The preferred method of purification is by HPLC.

Molecular weights of these peptides are determined using Quadrupole Electro Spray mass spectroscopy.

The synthesis process for the production of the NP derivatives of the present invention will vary widely, depending upon the nature of the various elements, i.e., the sequence of the NP peptide, the linking group and the reactive entity, comprised in the NP derivative. The synthetic procedures are selected to ensure simplicity, high yields and repetitivity, as well as to allow for a highly purified product. Normally, the chemically reactive entity will be coupled at the last stage of the synthesis, for example, with a carboxyl group, esterification to form an active ester. Specific methods for the production of the embodiment of NP derivatives of the present invention are described below.

It is imperative that the chemically reactive entity be placed at a site to allow the peptide to covalently bond to the blood component while retaining a substantial proportion, if not all, activity and/or beneficial effects of the corresponding NP peptide.

It is preferred to attach the reactive group at a site along the peptidic sequence of the NP peptide selected so as to not interfere with the binding activity and the pharmacologic activity of the NP peptide. In vitro assays may be used to select the best site to attach the reactive group.

The following examples are provided to illustrate preferred embodiments of the invention and shall by no means be construed as limiting its scope. Unless indicated otherwise, optically active protected amino acids in the L-configuration were used.

### PEPTIDE DERIVATIVE SYNTHESIS EXAMPLES

The synthesis of the present natriuretic peptides and derivatives thereof was performed using an automated solid-phase procedure on a Symphony™ peptide synthesizer with manual intervention during the generation of the Natriuretic derivatives. The synthesis was performed on Fmoc-protected Ramage Amide Linker™ resin using Fmoc-protected amino acids. Coupling was achieved by using O-benzotriazol-1-yl-N,N,N′,N′-tetramethyl-uronium hexafluorophosphate (HBTU) as activator in N,N-dimethylformamide (DMF) solution and diisopropylethylamine (DIEA) as base. The Fmoc protective group was removed using 20% piperidine/DMF. When needed, a Boc-protected amino acid was used at the N-terminus in order to generate the free N_{α}-terminus after the peptide was cleaved from the resin. All amino acids used during the synthesis possessed the L-stereochemistry unless otherwise stated. Glass reaction vessels were Sigmacoted™ and used during the synthesis.

In order to make easier the relation between the examples and the formula, it can be noted that the NP peptides and NP derivatives prepared in Examples 1 to 20 comprise NP peptides in accordance with the first preferred embodiment of the present invention, and ones prepared in Examples 21 to 57 comprise NP peptides in accordance with the second preferred embodiment of the present invention. It should be understood that a peptidic bond links the last amino acid on the first line and the first amino acid on the second line for each sequence given in the examples. It should also be understood that the line between the two cysteines in each sequence illustrated in the present application represents a direct disulfide bridge that forms a loop in the sequence.

### Example 1

| | |
|---|---|
| Ser-Leu-Arg-Arg-Ser-Ser-Cys-Phe-Gly-Gly-Arg-Met-Asp-Arg-Ile-Gly- | SEQ ID NO: 1 |
| \| | |
| Ala-Gln-Ser-Gly-Leu-Gly-Cys-Asn-Ser-Phe-Arg-Tyr-CONH₂ | |

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Trt)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 20% mixture of acetonitrile in water (0.1% TFA) and lyophilized to generate the corresponding crude material used in the purification process.

Step 3: The resulting peptide fully deprotected and was purified according to the standard purification procedure detailed herein below. The desired fractions were collected pooled together and lyophilised.

Step 4: The lyophilate of step 3 was placed in 2.5 mL AcOH/H₂O (1:1). Then iodine (I₂) (6 eq.) was added and followed by mass spectrometry (LC/MS) to monitor the reaction. The solution was stirred at room temperature for 12 hours. After the elapsed time, a solution of vitamine C (ascorbic acid 1M) was added. The precipitate was filtered out and the filtrate was lyophilized.

Step 5: The lyophilate of Step 4 was purified using standard purification procedure (detailed herein below).

### Example 2

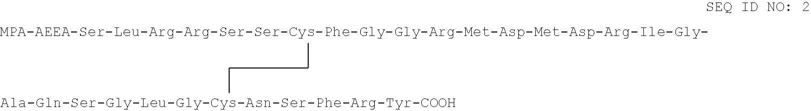

Step 1: Native Atrial Natriuretic peptide (provided by Phoenix Pharmaceuticals Inc., Belmont, Calif., USA, catalog number 0056) was placed in DMF. To the solution was added MPA-AEEA-COO(Su) and N-Methyl Morpholine. The solution was stirred for 6 hours and then the solution was diluted (1:1) with water and it was purified according to the standard methodology.

### Example 3

| | |
|---|---|
| MPA-AEEA-Ser-Leu-Arg-Arg-Ser-Ser-Cys-Phe-Gly-Gly-Arg-Met-Asp-Arg- | SEQ ID NO: 3 |
| \| | |
| Ile-Gly-Ala-Gln-Ser-Gly-Leu-Gly-Cys-Asn-Ser-Phe-Arg-Tyr-CONH₂ | |

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 20% mixture of acetonitrile in water (0.1% TFA) and lyophilized to generate the corresponding crude material used in the purification process.

Step 3: The resulting peptide fully deprotected, except for the Acm groups which remained attached to the thiol portion of the cysteine, and was purified according to the standard purification procedure detailed herein below. The desired fractions were collected pooled together and lyophilised.

Step 4: The lyophilate of step 3 was placed in neat TFA (trifluoroacetic acid) (1 mg/mL). Then anisole (100 eq.) was added followed by methyltrichlorosilane (10 eq.) and finally by diphenylsulphoxide (100 eq.). The solution was stirred at room temperature for 18 hours. After the elapsed time, the solution was placed in a separatory funnel with 2N Acetic acid (1 mL/mg of peptide) and cold ether (5 mL/mL of TFA). After multiple extractions, the desired cyclised peptides, present in the aqueous solution, were collected, combined together and lyophilised.

Step 5: The lyophilate of Step 4 was purified using standard purification procedure (detailed herein below).

### Example 4

| | |
|---|---|
| MPA-Ser-Leu-Arg-Arg-Ser-Ser-Cys-Phe-Gly-Gly-Arg-Met-Asp-Arg-Ile- | SEQ ID NO: 4 |
| \| | |
| Gly-Ala-Gln-Ser-Gly-Leu-Gly-Cys-Asn-Ser-Phe-Arg-Tyr-CONH₂ | |

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Fmoc-Ser(tBu)-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 5

| | |
|---|---|
| Ser-Leu-Arg-Arg-Ser-Ser-Cys-Phe-Gly-Gly-Arg-Met-Asp-Arg-Ile-Gly- | SEQ ID NO: 5 |
| \| | |
| Ala-Gln-Ser-Gly-Leu-Gly-Cys-Asn-Ser-Phe-Arg-Tyr-Lys(AEEA-MPA)-CONH₂ | |

Step 1: Solid phase peptide synthesis was carried out on a 100 μmole scale. The following protected amino acids were sequentially added to resin: Fmoc-Lys(Aloc)-OH, Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1):2.5% NMM (v:v):5% AcOH (v:v) for 2 h. The resin is then washed with CHCl₃ (6×5 mL), 20% AcOH in DCM (6×5 mL), DCM (6×5 mL), and DMF (6×5 mL).

Step 3: The synthesis was then re-automated for the addition of the Fmoc-AEEA-OH. After coupling the Fmoc protecting group was removed using 20% piperidine. Finally, 3-maleimidopropionic acid was coupled to the peptide on resin using standard coupling conditions. Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 40% mixture of acetonitrile in water (0.1% TFA) and lyophilized to generate the corresponding crude material used in the purification process.

Step 5: The resulting peptide fully deprotected, except for the Acm groups which remained attached to the thiol portion of the cysteine, was purified according to the standard purification procedure. The desired fractions were collected pooled together and lyophilised.

Step 6: The lyophilate of step 3 was placed in neat TFA (trifluoroacetic acid) (1 mg/mL). Then anisole (100 eq.) was added followed by methyltrichlorosilane (10 eq.) and finally by diphenylsulphoxide (100 eq.). The solution was stirred at room temperature for 18 hours. After the elapsed time, the solution was placed in a separatory funnel with 2N Acetic acid (1 mL/mg of peptide) and cold ether (5 mL/mL of TFA). After multiple extractions the aqueous solution were collected, combined toghether and lyophilised.

Step 7: The lyophilate of Step 4 was purified using standard purification methodology.

### Example 6

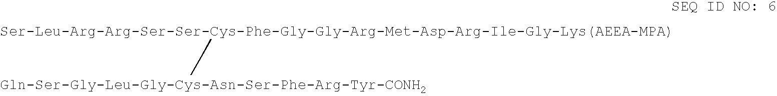

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Lys(Aloc)-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 5.

### Example 7

| | |
|---|---|
| Ser-Leu-Arg-Arg-Ser-Ser-Cys-Phe-Gly-Gly-Arg-Lys(AEEA-MPA)-Asp-Arg-Ile-Gly- | SEQ ID NO: 7 |
| \| | |
| Ala-Gln-Ser-Gly-Leu-Gly-Cys-Asn-Ser-Phe-Arg-Tyr-CONH₂ | |

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Lys(Aloc)-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 5.

### Example 8

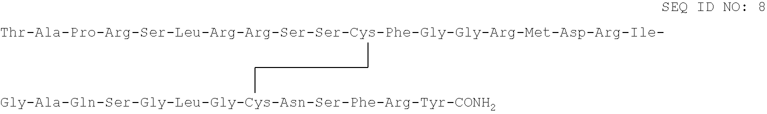

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Fmoc-Ser(tBu)-OH, Fmoc-Arg(Pbf)-OH, Fmoc-Pro-OH, Fmoc-Ala-OH, Boc-Thr(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 3.

### Example 9

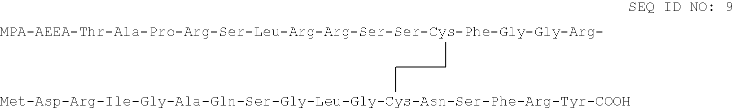

Step 1: Same as Step 1 in example 2 using urodilatin as starting material. Urodilatin is provided by Bachem, Torance, Calif., USA, catalog number H-3046.1000.

### Example 10

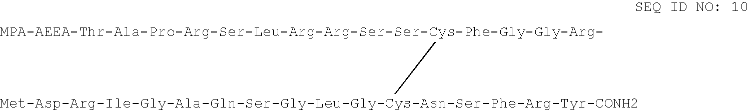

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Fmoc-Ser(tBu)-OH, Fmoc-Arg(Pbf)-OH, Fmoc-Pro-OH, Fmoc-Ala-OH, Fmoc-Thr(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 3.

### Example 11

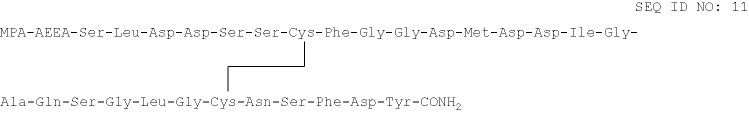

Step 1: Solid phase peptide synthesis was carried out on a 100 μmole scale. The following protected amino acids were sequentially added to resin: Fmoc-Tyr(tBu)-OH, Fmoc-Asp(tBu)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Asp(tBu)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Asp(tBu)-OH, Fmoc-Asp(tBu)-OH, Fmoc-Leu-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 12

SEQ ID NO: 12
Ser-Leu-Asp-Asp-Ser-Ser-Cys-Phe-Gly-Gly-Asp-Met-Asp-Arg-Ile-Gly-
|
Ala-Gln-Ser-Gly-Leu-Gly-Cys-Asn-Ser-Phe-Asp-Tyr-CONH₂

Step 1: Solid phase peptide synthesis was carried out on a 100 μmole scale. The following protected amino acids were sequentially added to resin: Fmoc-Tyr(tBu)-OH, Fmoc-Asp(tBu) OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Asp(tBu)-OH, Fmoc-Gly-OH, Fmoc-Gly-OH, Fmoc-Phe-OH, Fmoc-Cys(Acm)-OH, Fmoc-Ser(tBu)-OH, Fmoc-Ser(tBu)-OH, Fmoc-Asp(tBu)-OH, Fmoc-Asp(tBu)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 13

SEQ ID NO: 13
Cys-Phe-Gly-Gly-Arg-Ile-Asp-Arg-Ile-Gly-Ala-Gln-Ser-Gly-Leu-Gly-
|
Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Ile-OH, Fmoc-Arg(Pbf)-OH, Fmoc-Gly-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 14

| | |
|---|---|
| MPA-AEEA-Cys-Phe-Gly-Gly-Arg-Ile-Asp-Arg-Ile-Gly-Ala-Gln-Ser- | SEQ ID NO: 14 |
| \| | |
| Gly-Leu-Gly-Cys-Asn-Ser-Phe-Arg-Tyr-CONH₂ | |

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Ile-OH, Fmoc-Arg(Pbf)-OH, Fmoc-Gly-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 15

SEQ ID NO: 15
Ser-Ser-Cys-Phe-Gly-Gly-Arg-Met-Asp-Arg-Ile-Gly-Ala-Gln-Ser-Gly-
|
Leu-Gly-Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc Cys(Acm)-OH, Fmoc-Ser(tBu)-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 16

SEQ ID NO: 16
MPA-AEEA-Ser-Ser-Cys-Phe-Gly-Gly-Arg-Met-Asp-Arg-Ile-Gly-Ala-Gln-
|
Ser-Gly-Leu-Gly-Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-Ser(tBu)-OH, Boc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 17

SEQ ID NO: 17
Cys-Phe-Gly-Gly-Arg-Met-Asp-Arg-Ile-Gly-Ala-Gln-Ser-Gly-Leu-Gly-
|
Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (D) for 20 minutes.

Step 2-5: The steps were performed in the same manner as Example 3.

### Example 18

SEQ ID NO: 18
MPA-AEEA-Cys-Phe-Gly-Gly-Arg-Met-Asp-Arg-Ile-Gly-Ala-Gln-Ser-Gly-
|
Leu-Gly-Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 19

SEQ ID NO: 19

Ser-Leu-Arg-Arg-Ser-Ser-Cys-(N-Methyl-Phe)-Gly-Gly-Arg-Met-Asp-Arg-Ile-Gly-
|
Ala-Gln-Ser-Gly-Leu-Gly-Cys-Asn-Ser-Phe-Arg-Tyr-CONH₂

Step 1: Solid phase peptide synthesis was carried out on a 100 μmole scale. The following protected amino acids were sequentially added to resin: Fmoc-Tyr(tBu)-OH, Fmoc-Arg(Pbf)-OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-(N-Methyl)-Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 20

SEQ ID NO: 20
MIPA-AEEA-Ser-Leu-Arg-Arg-Ser-Ser-Cys-(N-Methyl-Phe)-Gly-Gly-Arg-Met-Asp-
|
Arg-Ile-Gly-Ala-Gln-Ser-Gly-Leu-Gly-Cys-Asn-Ser-Phe-Arg-Tyr-CONH2

Step 1: Solid phase peptide synthesis was carried out on a 100 μmole scale. The following protected amino acids were sequentially added to resin: Fmoc-Tyr(tBu)-OH, Fmoc-Arg(Pbf) OH, Fmoc-Phe-OH, Fmoc-Ser(tBu)-OH, Fmoc-Asn(Trt)-OH, Fmoc-Cys(Acm)-OH, Fmoc-Gly-OH, Fmoc-Leu-OH, Fmoc-Gly-OH, Fmoc-Ser(tBu)-OH, Fmoc-Gln(Trt)-OH, Fmoc-Ala-OH, Fmoc-Gly-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Gly-OH, Fmoc-Gly-OH, Fmoc-(N-Methyl)Phe-OH, Fmoc-Cys(Acm)-OH, Fmoc-Ser(tBu)-OH, Fmoc-Ser(tBu)-OH, Fmoc-Arg(Pbf)-OH, Fmoc-Arg(Pbf)-OH, Fmoc-Leu-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5: The steps were performed in the same manner as Example 3.

### Example 21

SEQ ID NO: 21

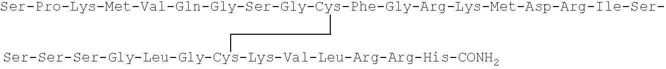

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 40% mixture of acetonitrile in water (0.1% TFA) and lyophilized to generate the corresponding crude material used in the purification process.

Step 3: The resulting peptide fully deprotected, except for the Acm groups which remained attached to the thiol portion of the cysteine, and was purified according to the standard purification procedure detailed herein below. The desired fractions were collected pooled together and lyophilised.

Step 4: The lyophilate of step 3 was placed in neat TFA (trifluoroacetic acid) (1 mg/mL). Then anisole (100 eq.) was added followed by methyltrichlorosilane (10 eq.) and finally by diphenylsulphoxide (100 eq.). The solution was stirred at room temperature for 18 hours. After the elapsed time, the solution was placed in a separatory funnel with 2N Acetic acid (1 mL/mg of peptide) and cold ether (5 mL/mL of TFA). After multiple extractions the aqueous solution were collected, combined together and lyophilised.

Step 5: The lyophilate of Step 4 was purified using standard purification procedure (detailed herein below).

### Example 22

SEQ ID NO: 22
Ser-Gly-Cys-Phe-Gly-Arg-Lys-Met-Asp-Arg-Ile-Ser-Ser-Ser-Ser-Gly-
|
Leu-Gly-Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 23

SEQ ID NO: 23
Cys-Phe-Gly-Gly-Arg-Ile-Asp-Arg-Ile-Gly-Ala-Gln-Ser-Gly-Leu-Gly-
|
Cys-Asn-Ser-Phe-Arg-Tyr-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 24

SEQ ID NO: 24
Ser-Gly-Cys-Phe-Gly-Arg-Lys-Met-Asp-Arg-Ile-Ser-Ser-Ser-Ser-Gly-
|
Leu-Gly-Cys-Lys-Val-Leu-Arg-Arg-His-Lys(AEEA-MPA)-CONH₂

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Boc-Ser(tBu)-OH, They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1):2.5% NMM (v:v):5% AcOH (v:v) for 2 h. The resin is then washed with CHCl₃ (6×5 mL), 20% AcOH in DCM (6×5 mL), DCM (6×5 mL), and DMF (6×5 mL).

Step 3: The synthesis was then re-automated for the addition of the Fmoc-AEEA-OH. After coupling the Fmoc protecting group was removed using 20% piperidine. Finally, 3-maleimidopropionic acid was coupled to the peptide on resin using standard coupling conditions. Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 40% mixture of acetonitrile in water (0.1% TFA) and lyophilized to generate the corresponding crude material used in the purification process.

Step 5: The resulting peptide fully deprotected, except for the Acm groups which remained attached to the thiol portion of the cysteine, was purified according to the standard purification procedure. The desired fractions were collected pooled together and lyophilised.

Step 6: The lyophilate of step 3 was placed in neat TFA (trifluoroacetic acid) (1 mg/mL). Then anisole (100 eq.) was added followed by methyltrichilorosilane (10 eq.) and finally by diphenylsulphoxide (100 eq.). The solution was stirred at room temperature for 18 hours. After the elapsed time, the solution was placed in a separatory funnel with 2N Acetic acid (1 mL/mg of peptide) and cold ether (5 mL/mL of TFA). After multiple extractions the aqueous solution were collected, combined toghether and lyophilised.

Step 7: The lyophilate of Step 4 was purified using standard purification methodology.

### Example 25

SEQ ID NO: 25

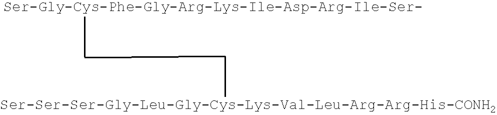

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 26

SEQ ID NO: 26

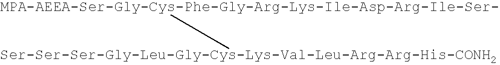

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 27

SEQ ID NO: 27
Ser-Gly-Cys-Phe-Gly-Arg-Lys-Ile-Asp-Arg-Ile-Ser-Ser-Ser-Ser-Gly-
|
Leu-Gly-Cys-Lys-Val-Leu-Arg-Arg-His-Lys(AEEA-MPA)-CONH₂

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7 The steps were performed in the same manner as Example 24.

### Example 28

SEQ ID NO: 28

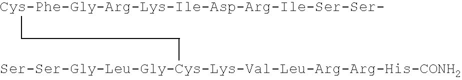

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 29

SEQ ID NO: 29
MPA-AEEA-Cys-Phe-Gly-Arg-Lys-Ile-Asp-Arg-Ile-Ser-Ser-Ser-Ser-Gly-
|
Leu-Gly-Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 30

SEQ ID NO: 30
Cys-Phe-Gly-Arg-Lys-Ile-Asp-Arg-Ile-Ser-Ser-Ser-Ser-Gly-Leu-Gly-
|
Cys-Lys-Val-Leu-Arg-Arg-His-Lys(AEEA-MIPA)-CONH₂

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7 The steps were performed in the same manner as Example 24.

### Example 31

SEQ ID NO: 31
Cys-Phe-Gly-Arg-Lys-Met-Asp-Arg-Ile-Ser-Ser-Ser-Ser-Gly-Leu-Gly-
|
Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 32

SEQ ID NO: 32
MPA-AEEA-Cys-Phe-Gly-Arg-Lys-Met-Asp-Arg-Ile-Ser-Ser-Ser-Ser-Gly-
|
Leu-Gly-Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 33

SEQ ID NO: 33
Cys-Phe-Gly-Arg-Lys-Met-Asp-Arg-Ile-Ser-Ser-Ser-Ser-Gly-Leu-Gly-
|
Cys-Lys-Val-Leu-Arg-Arg-His-Lys(AEEA-MPA)-CONH₂

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using 0-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7: The steps were performed in the same manner as Example 24.

### Example 34

SEQ ID NO: 34
Cys-(N^{a}Me-Phe)-Gly-Arg-Lys-Met-Asp-Arg-Ile-Ser-Ser-Ser-Ser-Gly-Leu-Gly-
|
Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-N-Methyl-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 35

SEQ ID NO: 35
MPA-AEEA-Cys-(N^{a}Me-Phe)-Gly-Arg-Lys-Met-Asp-Arg-Ile-Ser-Ser-Ser-Ser-Gly-
|
Leu-Gly-Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-N^{α}-Methyl-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 36

SEQ ID NO: 36
Cys-(N^{a}Me-Phe)-Gly-Arg-Lys-Met-Asp-Arg-Ile-Ser-Ser-Ser-Ser-Gly-Leu-Gly-
|
Cys-Lys-Val-Leu-Arg-Arg-His-Lys-(AEEA-MPA)-CONH₂

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-N^{α}-Methlyl-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7: The steps were performed in the same manner as Example 24.

### Example 37

SEQ ID NO: 37

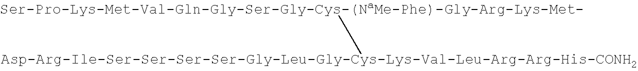

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-N-Methlyl-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 38

SEQ ID NO: 38

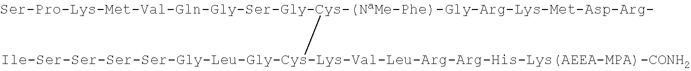

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-N^{α}-Methlyl-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Fmoc-Ser(tBu)-OH. Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7: The steps were performed in the same manner as Example 24.

### Example 39

SEQ ID NO: 39

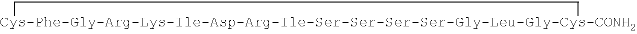

Step 1: Solid phase peptide synthesis was carried out on a 100 μmole scale. The following protected amino acids were sequentially added to resin: Fmoc-Cys(Acm)-OHa, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 40

SEQ ID NO: 40
MPA-AEEA-Cys-Phe-Gly-Arg-Lys-Ile-Asp-Arg-Ile-Ser-Ser-Ser-Ser-Gly-
Leu-Gly-Cys-CONH₂

Step 1: Solid phase peptide synthesis was carried out on a 100 μmole scale. The following protected amino acids were sequentially added to resin: Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 41

SEQ ID NO: 41
Cys-Phe-Gly-Arg-Lys-Ile-Asp-Arg-lIe-Ser-Ser-Ser-Ser-Gly-Leu-Gly-
|
Cys-Lys(AEEA-MIPA)-CONH₂

Step 1: Solid phase peptide synthesis was carried out on a 100 μmole scale. The following protected amino acids were sequentially added to resin: Fmoc-Lys(Aloc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7 The steps were performed in the same manner as Example 24.

### Example 42

SEQ ID NO: 42

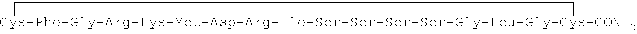

Step 1: Solid phase peptide synthesis was carried out on a 100 μmole scale. The following protected amino acids were sequentially added to resin: Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 43

SEQ ID NO: 43
MPA-AEEA-Cys-Phe-Gly-Arg-Lys-Met-Asp-Arg-Ile-Ser-Ser-Ser-Ser-Gly-
|
Leu-Gly-Cys-CONH₂

Step 1: Solid phase peptide synthesis was carried out on a 100 μmole scale. The following protected amino acids were sequentially added to resin: Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 44

SEQ ID NO: 44
Cys-Phe-Gly-Arg-Lys-Met-Asp-Arg-Ile-Ser-Ser-Ser-Ser-Gly-Leu-Gly-
|
Cys-Lys(AEEA-MPA)-CONH₂

Step 1: Solid phase peptide synthesis was carried out on a 100 μmole scale. The following protected amino acids were sequentially added to resin: Fmoc-Lys(Aloc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Boc-Cys(Acm)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7: The steps were performed in the same manner as Example 24.

### Example 45

SEQ ID NO: 45
Ser-Pro-Lys-Ile-Val-Gln-Gly-Ser-Gly-Cys-Phe-Gly-Arg-Lys-Ile-Asp-
|
Arg-Ile-Ser-Ser-Ser-Ser-Gly-Leu-Gly-Cys-Lys-Val-Leu-Arg-Arg-His-CONH₂

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Ile-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 46

SEQ ID NO: 46

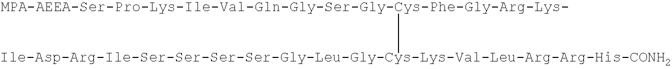

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Ile-OH, Fmoc-Lys(Boc)-OH, Fmoc-Pro-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 47

SEQ ID NO: 47

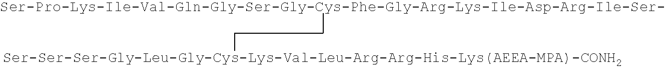

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Ile-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Ile-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 24.

### Example 48

SEQ ID NO: 48

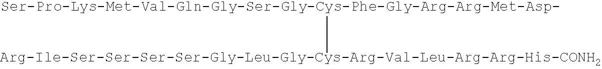

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Arg(Pbf)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 49

SEQ ID NO: 49

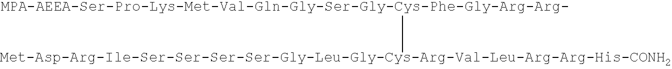

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Arg(Pbf)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 50

SEQ ID NO: 50

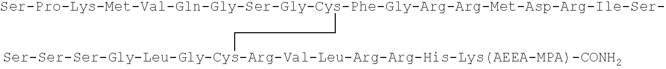

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Arg(Pbf)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Arg(Pbf)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 24.

### Example 51

SEQ ID NO: 51

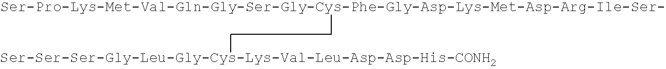

Step 1: Solid phase peptide synthesis was carried out on a 100 μmole scale. The following protected amino acids were sequentially added to resin: Fmoc-His(Trt)-OH, Fmoc-Asp(tBu)-OH, Fmoc-Asp(tBu)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Asp(tBu)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 52

| | |
|---|---|
| MPA-AEEA-Ser-Pro-Lys-Met-Val-Gln-Gly-Ser-Gly-Cys-Phe-Gly-Asp- | SEQ ID NO:52 |
| \| | |
| Lys-Met-Asp-Arg-Ile-Ser-Ser-Ser-Ser-Gly-Leu-Gly-Cys-Lys-Val-Leu- | |
| Asp-Asp-His-CONH₂ | |

Step 1: Solid phase peptide synthesis was carried out on a 100 μmole scale. The following protected amino acids were sequentially added to resin: Fmoc-His(Trt)-OH, Fmoc-Asp(tBu)-OH, Fmoc-Asp(tBu)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Asp(tBu)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 53

SEQ ID NO: 53

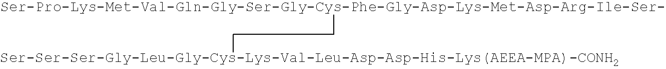

Step 1: Solid phase peptide synthesis was carried out on a 100 μmole scale. The following protected amino acids were sequentially added to resin: Fmoc-Lys(Aloc)-OH Fmoc-His(Trt)-OH, Fmoc-Asp(tBu)-OH, Fmoc-Asp(tBu)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Asp(tBu)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2 to 7 The steps were performed in the same manner as Example 24.

### Example 54

SEQ ID NO: 54

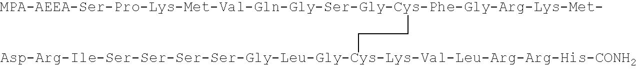

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Fmoc-Ser(tBu)-OH, Fmoc-AEEA-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-5 The steps were performed in the same manner as Example 21.

### Example 55

SEQ ID NO: 55

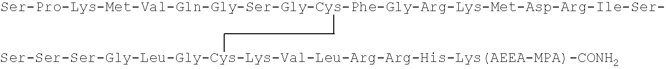

Step 1: Solid phase peptide synthesis was carried out on a 100 μmole scale. The following protected amino acids were sequentially added to resin: Fmoc-Lys(Aloc)-OH, Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 24.

### Example 56

| | |
|---|---|
| Ser-Pro-Lys-Met-Val-Gln-Gly-Ser-Gly-Cys-Phe-Gly-Arg-Lys-Lys(AEEA-MPA)-Asp- | SEQ ID NO:56 |
| \| | |
| Arg-Ile-Ser-Ser-Ser-Ser-Gly-Leu-Gly-Cys-Lys-Val-Leu-Arg-Arg-His-CONH₂ | |

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Ser(tBu)-OH, Fmoc-Ile-OH, Fmoc-Arg(Pbf)-OH, Fmoc-Asp(tBu)-OH, Fmoc-Lys(Aloc)-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 24.

### Example 57

SEQ ID NO: 57

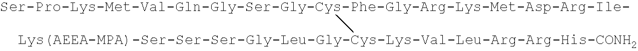

Step 1: Solid phase peptide synthesis was carried out on a 100 μmole scale. The following protected amino acids were sequentially added to resin: Fmoc-His(Trt)-OH, Fmoc-Arg(Pbf)-OH, Fmoc-Arg(Pbf)-OH, Fmoc-Leu-OH, Fmoc-Val-OH, Fmoc-Lys(Boc)-OH, Fmoc-Cys(Acm)-OH, Fmoc-Gly-OH, Fmoc-Leu-OH, Fmoc-Gly-OH, Fmoc-Ser(tBu)-OH, Fmoc-Ser(tBu)-OH, Fmoc-Ser(tBu)-OH, Fmoc-Lys(Aloc)-OH, Fmoc-Ile-OH, Fmoc-Arg(Pbf)-OH, Fmoc-Asp(tBu)-OH, Fmoc-Met-OH, Fmoc-Lys(Boc)-OH, Fmoc-Arg(Pbf)-OH, Fmoc-Gly-OH, Fmoc-Phe-OH, Fmoc-Cys(Acm)-OH, Fmoc-Gly-OH, Fmoc-Ser(tBu)-OH, Fmoc-Gly-OH, Fmoc-Gln(Trt)-OH, Fmoc-Val-OH, Fmoc-Met-OH, Fmoc-Lys(Boc)-OH, Fmoc-Pro-OH, Boc-Ser(tBu)-OH. They were dissolved in N,N-dimethylformamide (DMF) and, according to the sequence, activated using O-benzotriazol-1-yl-N,N,N′,N′-tetramethyl-uronium hexafluorophosphate (HBTU) and diisopropylethylamine (DIEA). Removal of the Fmoc protecting group was achieved using a solution of 20% (V/V) piperidine in N,N-dimethylformamide (DMF) for 20 minutes.

Step 2-7 The steps were performed in the same manner as Example 24.

Purification Procedure of the Synthetised Derivative

Each compound was purified by preparative reversed phase HPLC, using a Varian (Dynamax) preparative binary HPLC system. The purification was performed using a Phenomenex Luna 10μ phenyl-hexyl, 50 mm×250 mm column (articles 10μ) equilibrated with a water/TFA mixture (0.1% TFA in H₂O (solvent A) and acetonitrile/TFA (0.1% TFA in CH₃CN (solvent B). Fractions containing peptide were detected by UV absorbance (Varian Dynamax UVD II) at 214 nm. Table 2 shows the retention time of compounds that are NP peptides and derivatives according to the present invention.

TABLE 2

| Compound | Retention Time |
|---|---|
| Example 1 | 27.0^{A} |
| Example 2 | 13.0^{B} |
| Example 3 | 28.1^{A} |
| Example 4 | 27.9^{A} |
| Example 5 | 26.8^{A} |
| Example 6 | 26.8^{A} |
| Example 7 | 23.6^{A} |
| Example 8 | 9.0^{C} |
| Example 13 | 28.5^{A} |
| Example 14 | 31.0^{A} |
| Example 15 | 27.2^{A} |
| Example 16 | 29.8^{A} |
| Example 17 | 28.3^{A} |
| Example 18 | 31.0^{A} |
| Example 19 | 27.2^{A} |
| Example 20 | 28.8^{A} |
| Example 21 | 23.3^{A} |
| Example 54 | 24.9^{A} |
| Example 55 | 24.1^{A} |
| Example 56 | 23.5^{A} |
| Example 57 | 24.2^{A} |

The retention times annotated with A, B and C have been obtained with gradient of elution shown in Tables 3, 4 and 5 respectively.

TABLE 3

| Time (min) | Solvent A (%) | Solvent B (%) | Flow (ml/min) |
|---|---|---|---|
| 0 | 95.0 | 5.0 | 0.500 |
| 60 | 25.0 | 75.0 | 0.500 |
| 65 | 10.0 | 90.0 | 0.500 |
| 75 | 10.0 | 90.0 | 0.500 |
| 80 | 95.0 | 5.0 | 0.500 |
| 90 | 95.0 | 5.0 | 0.500 |

TABLE 4

| Time (min) | Solvent A (%) | Solvent B (%) | Flow (ml/min) |
|---|---|---|---|
| 0 | 80.0 | 20.0 | 0.500 |
| 20 | 30.0 | 70.0 | 0.500 |
| 21 | 10.0 | 90.0 | 0.500 |
| 26 | 10.0 | 90.0 | 0.500 |
| 27 | 80.0 | 20.0 | 0.500 |
| 32 | 80.0 | 20.0 | 0.500 |

TABLE 5

| Time (min) | Solvent A (%) | Solvent B (%) | Flow (ml/min) |
|---|---|---|---|
| 0 | 95.0 | 5.0 | 0.500 |
| 3 | 85.0 | 75.0 | 0.500 |
| 18 | 65.0 | 90.0 | 0.500 |
| 19 | 10.0 | 90.0 | 0.500 |
| 24 | 10.0 | 5.0 | 0.500 |
| 25 | 95.0 | 5.0 | 0.500 |
| 105 | 95.0 | 5.0 | 0.500 |

Table 6 shows the predicted molecular weight (Predicted) and measured molecular weight (Measured) of compounds that are NP peptides and derivatives according to the present invention. All the molecular weights are expressed in g/mol. Molecular weight has been measured by Quadrupole Electro Spray mass spectroscopy. The predicted molecular weight has been established by addition of the theoretical mass of each atom. The differences between the predicted molecular weight and the measured molecular weight are negligible and indicate that the compounds synthesized are the desired compounds.

TABLE 6

| Compound | Predicted | Measured |
|---|---|---|
| Example 1 | 3077.5 | 3078.5 |
| Example 2 | 3374.6 | 3377.0 |
| Example 3 | 3373.6 | 3377.1 |
| Example 4 | 3228.5 | 3230.3 |
| Example 5 | 3501.7 | 3504.0 |
| Example 6 | 3430.6 | 3432.5 |
| Example 7 | 3370.6 | 3372.3 |
| Example 8 | 3502.7 | 3504.5 |
| Example 13 | 2373.1 | 2374.0 |
| Example 14 | 2669.2 | 2670.1 |
| Example 15 | 2565.1 | 2566.0 |
| Example 16 | 2861.2 | 2862.2 |
| Example 17 | 2391.1 | 2392.1 |
| Example 18 | 2687.2 | 2688.2 |
| Example 19 | 3091.5 | 3092.8 |
| Example 20 | 3387.6 | 3388.9 |
| Example 21 | 3460.8 | 3462.7 |
| Example 54 | 3756.9 | 3758.9 |
| Example 55 | 3885.0 | 3887.3 |
| Example 56 | 3753.9 | 3755.9 |

Determination of the Efficiency of Cyclisation of the Peptide

Cyclisation is obtained by reduction of the thiol group of both cysteine residues of the peptide so as to form an intramolecular disulphide bridge and details of the process are in the specification and are exemplified in Step 4 of Example 1 and in Step 4 of Example 3. In order to determine that the peptide has been successfully cyclised, an Ellman test was performed on the final cyclised material as taught in G. L. Ellman, Arch. Biochem. Biophys., 82 (70) 1959 and G. L. Ellman, Biochem. Pharmacol., 7 (68) 1961. The Ellman test allows determination of thiol groups that would not form disulphide bridges. The absence of free thiol groups indicates that the cyclisation was successful.

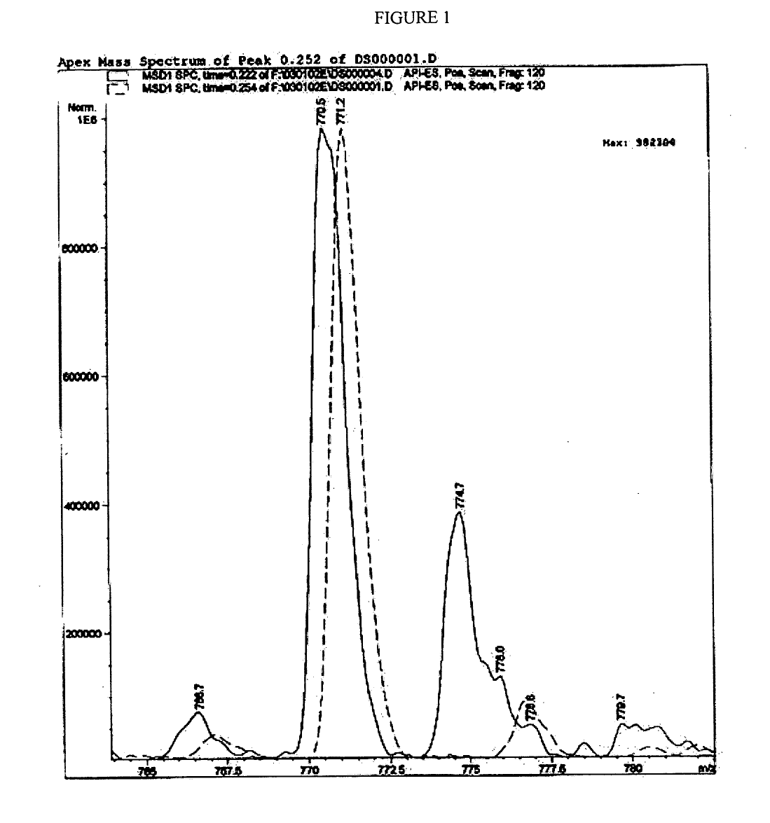

Also, analysis by LC/MS allows comparison of the intermediate of synthesis obtained before the step of cyclisation and the final product obtained after the cyclisation step. FIG. 1 show in superposition the LC/MS spectrums of the intermediates of synthesis of the compound of Example 1 before cyclisation illustrated in dotted line (---) and the corresponding final products after cyclisation illustrated in continuous line (-), wherein the cyclisation was performed with iodine as exemplified in Step 4 of Example 1. It can be seen that the intermediates have a molecular ion fragment of 771.2 (M+4) that corresponds to a mass of 3080.8 and the final products have a molecular ion fragment of 770.5 (M+4) that corresponds to a mass of 3078.0. The reduction of the mass of 2.8 results from the loss of two hydrogens during the formation of the disulphide bridge. The sharpness of the peaks of the linear intermediates and the cyclic final products indicate that all the intermediates were cyclised.

Moreover, no significant peak was seen at about 1232 (M+5) and/or 880.4 (M+7) (not shown), which means that no dimer was synthesized; in other words no intermolecular disulphide bridge was generated.

[image]

In Vitro Conjugation

Preparation of ex vivo conjugates is used for in vitro tests of the derivative and for the purposes of subsequent in vivo administration of the conjugate. Therefore, the derivative is conjugated to a blood component. Preferably, the blood component is human serum albumin (HSA). In examples 22-23-24, HSA is provided by Cortex-Biochem™, San Leandro, Calif., USA.

### IN VITRO CONJUGATION EXAMPLES

### Example 58

Preparation of 1 mM of the compound of Example 3:HSA conjugates. In a 1500 μL Eppendorf™ tube, 450 μL of HSA 25% (g/100 ml) is dispensed, and using a variable speed vortex machine, the HSA solution is vortexed. While vortexing, 50 μL of the compound of Example 3, at a concentration of 10 mM in nanopure water, is added. The resulting solution is incubated at 37° C. for 4 hours, and stored at 20° C.

### Example 59

Preparation of 1 mM of the compound of Example 4:HSA conjugates. Conjugation to HSA is performed in the same manner as Example 58.

### Example 60

Preparation of 1 mM of the compound of Example 5:HSA conjugates. Conjugation to HSA is performed in the same manner as Example 58.

### Example 61

Preparation of 1 mM of the compound of Example 6:HSA conjugates. Conjugation to HSA is performed in the same manner as Example 58.

### Example 62

Preparation of 1 mM of the compound of Example 7:HSA conjugates. Conjugation to HSA is performed in the same manner as Example 58.

### Example 63

Preparation of 1 mM of the compound of Example 14:HSA conjugates. Conjugation to HSA is performed in the same manner as Example 58.

### Example 64

Preparation of 1 mM of the compound of Example 18:HSA conjugates. Conjugation to HSA is performed in the same manner as Example 58.

### Example 65

Prepare 1 mM of the compound of Example 54:HSA conjugates. Conjugation to HSA is performed in the same manner as Example 58.

### Example 66

Preparation of 1 mM of the compound of Example 55:HSA conjugates. Conjugation to HSA is performed in the same manner as Example 58.

### Example 67

Preparation of 1 mM of the compound of Example 56:HSA conjugates. Conjugation to HSA is performed in the same manner as Example 58.

### Example 68

Preparation of 1 mM of the compound of Example 57:HSA conjugates. Conjugation to HSA is performed in the same manner as Example 58.

Conjugate Purity Analysis

For analyzing the purity of the prepared conjugates, two tests are performed by liquid chromatography/mass spectrometry (LC/MS) (Electro Spray Ionization, Agilent HP 1100 Series): 1) quantifying the residual free derivatives with comparison to 1% derivative reference and 2) detecting the conjugates with comparison to HSA.

Conjugate Purity Results

The residual free derivative remaining in solution is:
Example 58: Conjugates of compound of Example 3 with HSA: 2.2%
Example 59: Conjugates of compound of Example 4 with HSA: 4.4%
Example 60: Conjugates of compound of Example 5 with HSA: 3.6%
Example 61: Conjugates of compound of Example 6 with HSA: <1%
Example 62: Conjugates of compound of Example 7 with HSA: <1%
Example 63: Conjugates of compound of Example 14 with HSA: 1.2%
Example 64: Conjugates of compound of Example 18 with HSA: 1.3%
Example 65: Conjugates of compound of Example 54 with HSA: 1.4%
Example 66: Conjugates of compound of Example 55 with HSA: 2.4%
Example 67: Conjugates of compound of Example 56 with HSA: 0.8%
Example 68: Conjugates of compound of Example 57 with HSA: 2.1%
   Conjugate Weight

Table 7 shows the predicted molecular weight (Predicted) and measured molecular weight (Measured) of conjugates of NP derivatives according to the present invention. All the molecular weights are expressed in g/mol. Molecular weight has been measured by Quadrupole Electro Spray mass spectroscopy. The predicted molecular weight has been established by addition of the theoretical mass of each atom. The differences between the predicted molecular weight and the measured molecular weight are negligible and indicate that the compounds synthesized are the desired compounds.

TABLE 7

| Conjugate | Predicted | Measured |
|---|---|---|
| Example 58 | 69854 | 69853 |
| Example 59 | 69709 | 69708 |
| Example 60 | 69949 | 69943 |
| Example 61 | 69878 | 69874 |
| Example 62 | 69818 | 69814 |
| Example 63 | 69118 | 69108 |
| Example 64 | 69136 | 69128 |
| Example 65 | 70204 | 70202 |
| Example 66 | 70332 | 70329 |
| Example 67 | 70201 | 70199 |
| Example 68 | 70245 | 70243 |

In Vitro Binding and Activity Assays

The potency of NP derivatives is evaluated as their ability to bind NPR receptors in guinea pig adrenal glands and to elevate cGMP levels in a rat primary lung fibroblasts assay. Others cell lines can be used to perform these in vitro assays such as aortic smooth muscle cells, glomeruli mesangial cells and adrenal cells. Human, rat, and ginea pig cell lines or other species cell lines can be used with a preference for human cell lines.

### IN VITRO BINDING ASSAYS EXAMPLES

Membranes for binding studies are prepared as follow. Adrenal glands were collected from anesthetized normal Duncan Hartley Guinea Pig and homogenized using a polytron in 50 mM Tris-HCl buffer containing 150 mM NaCl, 5 mM MgCl₂, 5 mM MnCl₂; pH 7.4 at 25° C. The homogenate was centrifuged for 10 minutes at 39,000×g (4° C.). The pellet was resuspended and washed. Finally, the membranes were resuspended in the same buffer supplemented with 1 mM Na₂EDTA+0.2% BSA. Protein concentration is measured using the BCA protein assay kit (Pierce). The binding assay is done by incubation of membranes with 0.016 nM ¹²⁵I-rANF and increasing concentrations of either NP peptides or NP derivatives (10⁻⁵-10⁻¹¹ M) for 60 minutes at 4° C. All assays were done in duplicate. Separation of bound and free radioactive rANF was achieved by rapid filtration through polyethylenimine-treated Whatman GF/C filters soaked in assay buffer. Filters were washed, dried and counted for radioactivity in a gamma-counter.

Binding assays results of the NP derivatives comprising NP peptides of formula I are presented on FIG. 2 and the binding assays results of the NP derivatives comprising NP peptides based on formula II are presented on FIG. 3.

In FIG. 2, “Native ANP” is the peptide having the human ANP sequence that has been synthesized in our laboratories (see Example 1) and “hANP” is the commercial peptide provided by Phoenix Pharmaceuticals Inc., Belmont, Calif., USA, and catalogue number 005-06. As it can be seen on FIG. 2, native ANP and commercial hANP both inhibited the binding of ¹²⁵I-ANF to the receptor in a concentration-dependent manner with apparent inhibition constants (Ki values) of 3.4×10⁻¹⁰M and 6.0×10⁻¹⁰M, respectively. Conjugates of NP derivatives of Examples 3 and 5 also inhibited the binding of ¹²⁵I-ANF to the receptor of adrenal glands in a concentration-dependent manner with apparent Ki values of 2.4×10⁻⁹M and 2.9×10⁻⁹M respectively. Conjugates of NP derivatives of Examples 6 and 7 had a lower binding affinity and avidity for the NPR receptors. The derivatives of Examples 6 and 7 are modified in the loop in comparison with the derivatives of Examples 3 and 5, which are modified at the N-terminus and C-terminus respectively.

[image]

Table 8 shows the concentrations at 50% of inhibition (EC50) and the inhibition constants (KI) that were calculated with the data from which originates the graph in FIG. 2.

TABLE 8

| NP Peptides and | | |
|---|---|---|
| Conjugates | EC50 (M) | KI |
| HANP | 6.7230 e−010 | 6.0340 e−010 |
| Native ANP | 3.8260 e−010 | 3.4330 e−010 |
| Example 3: HSA | 2.7060 e−009 | 2.4290 e−009 |
| Example 5: HSA | 3.2330 e−009 | 2.9020 e−009 |
| Example 6: HSA | 6.5110 e−007 | 5.8440 e−007 |
| Example 7: HSA | 5.4730 e−006 | 4.9110 e−006 |

In FIG. 3, “Native BNP” is the peptide having the human BNP sequence that has been synthesized in our laboratories (see Example 21). As it can be seen on FIG. 3, native BNP inhibited the binding of ¹²⁵I-ANF to the receptor in a concentration-dependent manner with an apparent inhibition constant (Ki value) of 4.8×10⁻⁹M. Conjugates of NP derivatives of Examples 54 and 55 also inhibited the binding of ¹²⁵I-ANF to the receptor of adrenal glands in a concentration-dependent manner with apparent Ki values of 1.5×10⁻⁸M and 5.5×10⁻⁸M respectively. Conjugates of NP derivatives of Examples 56 and 57 had a lower binding affinity and avidity for the NPR receptors. The derivatives of Examples 56 and 57 are modified in the loop in comparison with the derivatives of Examples 54 and 55, which are modified at the N-terminus and C-terminus respectively.

[image]

Table 9 shows the concentrations at 50% of inhibition (EC50) and the inhibition constants (KI) that were calculated with the data from which originates the graph in FIG. 3.

TABLE 9

| NP Peptides and | | |
|---|---|---|
| Conjugates | EC50 (M) | KI |
| HBNP | 5.4120 e−009 | 4.8570 e−009 |
| Example 54: HSA | 1.7080 e−008 | 1.5330 e−008 |
| Example 55: HSA | 6.0760 e−008 | 5.4530 e−008 |
| Example 56: HSA | 3.1200 e−007 | 2.8000 e−007 |
| Example 57: HSA | 2.8040 e−007 | 2.5160 e−007 |

### IN VITRO ACTIVITY ASSAYS EXAMPLES

For in vitro activity studies, a human cervix epithelial adenocarcinoma cell line was used. Hela cells express high levels of natriuretic peptide receptors with guanylate cyclase activity.

One day prior cGMP experiments, cells are seeded in 48-wells plate (5×10⁴ cells per well) and incubated overnight. The day of the experiment cells are washed twice in serum-free media and then incubated with or without NP derivatives or native ANP or BNP for one hour, in presence of 3-isobutyl-1-methylxanthine to prevent cGMP degradation. Incubation is terminated by removing the assay medium and by adding HCl to the cells for 10 minutes. The supernatants were then collected, centrifuged and cGMP levels are assessed using the direct cGMP EIA kit from Sigma.

All NP derivatives and conjugates were able to elevate cGMP in human Hela cells at concentration ranging from 10⁻⁶M to 10⁻⁹M, except for the conjugates of the derivatives of Examples 14, 18 and 56 as illustrated in FIGS. 4, 5 and 6. The EC50 (Effective Concentration of a drug that causes 50% of the maximum response) have been calculated for each NP derivative and conjugate and are listed in Table 10. As it can be seen from Table 10, the increase in cGMP is comparable to that obtained from native ANP and no significant (p<0.05) differences are observed between them, with exception for the conjugates Example 14:HSA, Example 18:HSA and Example 56:HSA. Assays were performed in duplicata and each compound was tested three times.

[image]

[image]

TABLE 10

| NP Peptides and | |
|---|---|
| Conjugates | EC50 (M) |
| Native ANP | 2.43 × 10⁻⁹ |
| Example 3: HSA | 1.73 × 10⁻⁸ |
| Example 4: HSA | 4.21 × 10⁻⁸ |
| Example 5: HSA | 3.67 × 10⁻⁸ |
| Example 13 | 2.72 × 10⁻⁸ |
| Example 14: HSA | >10⁻⁶ |
| Example 17 | 2.21 × 10⁻⁸ |
| Example 18: HSA | >10⁻⁶ |
| Native BNP | 1.99 × 10⁻⁸ |
| Example 54: HSA | 1.75 × 10⁻⁸ |
| Example 55: HSA | 1.43 × 10⁻⁸ |
| Example 56: HSA | >10⁻⁶ |
| Example 57: HSA | 3.36 × 10⁻⁸ |

Analysis of the Stability in Human Plasma

Stability of conjugates of NP peptides is tested in human plasma in comparison to the corresponding free NP peptides so as to show protection of the conjugated NP peptides against enzymatic degradation occurring in human plasma or to select the more stable NP derivatives. In the examples given below, the corresponding free NP peptide is human ANP, called “hANP” herein below, which was provided by Phoenix Pharmaceuticals Inc., Belmont, Calif., USA.

Conditions for the analysis of the stability in human plasma are as follow. 750 μL of human plasma (Biochemed Inc., Winchester, Va., USA) is poured in a 1500 μL Eppendorf Tube and 250 μL of NP conjugates or hANP 1 mM is added to the plasma in order to obtain a final concentration of 0.25 mM of conjugates or hANP. The solutions are mixed by vortexing and the timer is started. The solutions are incubated at 37° C. for 48 hours. An aliquot of 100 μL is removed at time zero, 2 hrs, 4 hrs, 8 hrs, 12 hrs, 24 hrs, and 48 hrs. Each aliquot is placed in a HPLC vial, snap freeze immediately on dry ice and stored at −80° C. until the LC/MS analysis.

The LC/MS elution gradient of the peptides and the conjugates are respectively shown in Table 11 and 12; where solvent A is water/TFA mixture (0.1% TFA in H₂O) and solvent B is acetonitrile/TFA (0.1% TFA in CH₃CN).

TABLE 11

| Time (min) | Solvent A (%) | Solvent B (%) | Flow (ml/min) |
|---|---|---|---|
| 0 | 80.0 | 20.0 | 0.500 |
| 20 | 40.0 | 60.0 | 0.500 |
| 25 | 10.0 | 90.0 | 0.500 |
| 30 | 10.0 | 90.0 | 0.500 |
| 35 | 80.0 | 20.0 | 0.500 |

TABLE 12

| Time (min) | Solvent A (%) | Solvent B (%) | Flow ml/min |
|---|---|---|---|
| 0 | 66.0 | 34.0 | 0.250 |
| 5 | 66.0 | 34.0 | 0.250 |
| 10 | 50.0 | 50.0 | 0.250 |
| 15 | 5.0 | 95.0 | 0.350 |
| 21 | 5.0 | 95.0 | 0.350 |
| 26 | 66.0 | 34.0 | 0.350 |

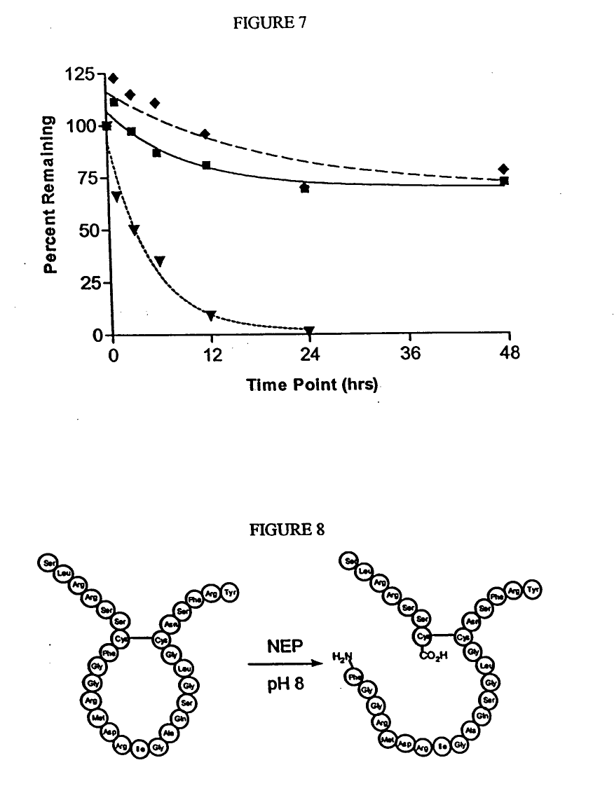

For each time point, results are reported as the percentage of peptide or conjugate peak height with respect to the total peak height of the sample. FIG. 7 shows the results for hANP (▾), conjugates of Example 58 (▪) and conjugates of Example 60 (♦).

[image]

It can be seen from FIG. 7, all the hANP is degraded after 24 hours of incubation in human plasma whereas more than 75% of the ANP conjugated with HSA is not degraded after 48 hours. The resulting half-life of hANP is about 4 hrs. The conjugates of Example 58 (▪) comprise an ANP sequence modified at the N-terminal (Example 23) and the conjugates of Example 60 (♦) comprise an ANP sequence modified at the C-terminal (Example 25). Both conjugates show similar results of stability in human plasma.

Analysis of the Stability Towards NEP Enzyme

Stability of conjugates of NP peptides is also tested in a NEP enzyme solution in comparison to the corresponding free NP peptides so as to show protection of the conjugated NP peptides against enzymatic degradation by NEP enzyme specifically. In the examples given below, the corresponding flee NP peptide is human ANP, called “hANP” herein below, which was provided by Phoenix Pharmaceuticals Inc., Belmont, Calif., USA.

Conditions for the analysis of the stability towards NEP enzyme degradation are as follow. The lyophilised enzymes contained in a vial of NEP enzyme (provided by Calbiochem/Novabiochem Corporation, San Diego, Calif., USA, product # 324762) are solubilized with 100 μL of 0.1 M Tris-HCl buffer pH 8.0. It was vortexed and sonnicated to ensure a complete dissolution of the enzymes. One vial contains between 800 and 950 U of enzymes. A solution of conjugates is prepared at 250 μM with 0.1 M Tris-HCl buffer pH 8.0. Ten parts of the solution of conjugates or hANP (250 μM) are added to 1 part of the NEP enzyme solution (as above prepared). The resulting solution is vortexed and incubated at 37° C. under mixing conditions for 48 hours. An aliquot of 50 μL is removed at time zero, 30 min, 1 hr, 2 hrs, 4 hrs, 8 hrs, 12 hrs, 24 hrs, and 48 hrs. Each aliquot is placed in a vial, snap freeze immediately on dry ice and stored at −80° C. until analysis.

The site of hydrolysis of NEP on the sequence of ANP is the Cys-Phe peptidic bond at the beginning of the loop, as illustrated in FIG. 8.

[image]

The BNP sequence is also cleaved by NEP at the same site, i.e. at the Cys-Phe peptidic bond at the beginning of the loop.

For detection of the non-hydrolysed NP peptide, radioimmunoassay (RIA) is performed using a commercial polyclonal antibody raised against human native ANP (Product # RGG-8798, Peninsula Laboratories Inc. Division of Bachem, San Carlos, Calif., USA).

For the radioimmunoassay, 50 μL of either NP conjugate calibration standards, quality control samples, or diluted test samples in assay buffer (0.05M phosphate buffer, pH 7.5, 0.08% sodium azide, 0.025M EDTA, and 0.1% gelatin) is added to the appropriately labeled 12×75 mm borosilicate glass test tubes. 50 μL of assay buffer is added to the NSB (Non Specific Binding) and zero-standard (Reference) tubes. Then, 300 μL of assay buffer is added to each NSB tube and 200 mL of this same buffer is added to each of the other 12×75 mm borosilicate glass test tubes. A volume of 100 μL of rabbit anti-ANP IgG working solution, at a concentration of 2 μg/mL in assay buffer, is then added to all tubes except TC (Total Counts) and NSB tubes. Tube contents are mixed and incubated overnight (16-24 hours) at approximately 4° C. On the second day, 100 μL of ¹²⁵I-hANP (approximately 20,000 cpm/100 μL) is added to all tubes. Tube contents are mixed and incubated overnight (16′-24 hours) at approximately 4° C. On the third day, 1000 μL of 0.6% charcoal in 0.05M phosphate buffer is added to all tubes except TC tubes. Tubes are mixed and incubated at approximately 4° C. for approximately 30 minutes. After incubation, all tubes except TC tubes are then centrifuged at 4000 rpm for approximately 30 minutes at approximately 4° C. Free antigen is separated from the bound antigen by decanting the supernatant. The supernatants (bound fractions) are then counted on a gamma counter (Packard Cobra II Auto-Gamma) for at least 2 minutes. The amount of [¹²⁵I]-labeled antigen bound to the antibody is inversely proportional to the concentration of antigen in the tubes.

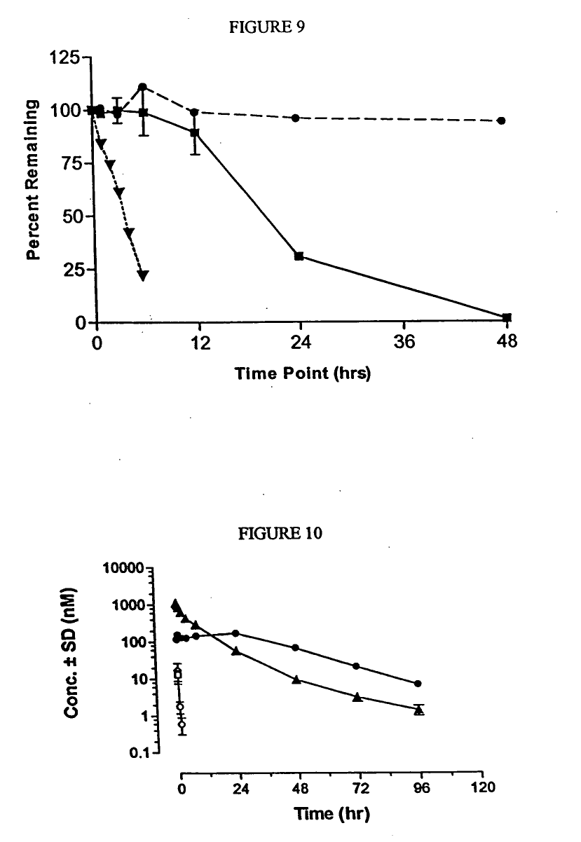

For each time point of the incubation with NEP enzyme, results are reported as the percentage of peptide or conjugate with respect to the total amount of the sample. FIG. 9 shows the results for hANP (▾), conjugates of Example 58 (▪) and capped HSA (●). “Capped HSA” is albumin with a cysteine residue bonded to it.

[image]

It can be seen from FIG. 9, most of the hANP is hydrolysed within 12 hours whereas the conjugated ANP (conjugates of Example 58) take about 48 hrs to be hydrolysed completely by NEP enzyme in the test conditions. In order to prove that the hydrolysis caused by NEP enzyme occurs in the ANP sequences and not in HSA, a control with capped HSA is used and shows that albumin is not (or almost not) subject to NEP hydrolysis.

Pharmacokinetic Studies

Pharmacokinetic studies of the derivatives are carried out in male Sprague-Dawley rats by subcutaneous (250 nmol/kg) or intravenous (50 nmol/kg) injection. Serial blood samples were taken at pre-dose and 5 min, 30 min, 1 hr, 2 hrs, 4 hrs, 8 hrs, 24 hrs, 48 hrs, 72 hrs and 96 hrs post-agent administration. Blood samples were collected into tubes containing K2-EDTA and aprotinin, then centrifuged to obtain plasma and kept frozen until analysis by radioimmunoassay (RIA). A commercial polyclonal antibody raised against human native ANP (Product # RGG-8798, Peninsula Laboratories Inc. Division of Bachem, San Carlos, Calif., USA) is used to detect the compounds. The assay sensitivity is 300 to 10 000 pM. Specific monoclonal antibodies need to be prepared and used for detecting each NP derivative that contains a NP peptide significatively different from the ANP and BNP. For derivatives of ANP and BNP, commercial antibodies are available. For the derivatives of NP peptide having a high homology with ANP or BNP, the commercially available antibodies may successflly be used in the RIA.

In FIG. 10, the bioavailability of free NP peptides is compared with the bioavailability of conjugated NP peptides. It can be seen that the conjugated ANP (conjugates of NP peptide of Example 3) administrered by intravenous injection (▴) or by subcutaneous injection (●) are still bioavailable after 96 hrs whereas free ANP (NP peptide of Example 3) administered by intravenous injection (□) or by subcutaneous injection (◯) are not present in the blood stream within 5 min.

[image]

In these rat studies, the half-life of the conjugated ANP (conjugates of Example 58) administered by intravenous injection (▴) or by subcutaneous injection (●) is 17.5±1.5 hours and 14.8±0.6 hours respectively. The half-life of free ANP (NP peptide of Example 3) administered by subcutaneous injection (◯) is 0.2±0.06 hour and the one for ANP administered by intravenous injection (□) could not be calculated since it was too short.

In Vivo Assays

Animal models of congestive heart failure are used to assess the optimal dose response, the duration of action and the most effective NP derivatives and NP conjugates. The two following animal models can be used to do so: the spontaneous hypertensive rats (SHR rats) and the pacing model in dogs (Muders and Elsner, Pharm Res, 2000). Since native BNP is known to have no activity in rats, the derivatives of NP peptides having a high homology with BNP are not tested in the SHR rats; therefore dogs' models or other models would be used.

SHR rats are genetically hypertensive rats, which develop significantly elevated systolic blood pressure (BP) by 4 weeks of age. As a consequence of sustained elevated blood pressure throughout their lifetimes, these rats develop congestive heart failure by around 1 year of age. In addition to high blood pressure, this model is also characterized by left ventricular hypertrophy and left ventricular fibrosis. SHR rats have been used previously in studies of the in vivo effects of atrial natriuretic peptide. Single doses of ANP analogues produced a temporary drop in BP, while continuous infusions were required to sustain a decrease in systolic BP (DeMay et. al. J Pharm Exper Therap, 1987).

The pacing model in dogs, involves the implantation of programmable cardiac pacemakers. After a surgical recovery period, the heart rate is increased incrementally from 180 to 240 beats/min over a 31 to 38 day period. This model allows for the study of different stages of heart failure, evolving from the normal heart, to asymptomatic left ventricular dysfunction, to overt congestive heart failure (Luchner et. al. Eur J Heart Failure, 2000). Characteristics of this model include increases of heart rate, increased cardiac filling pressure, low cardiac output, edema formation and activation of the sympathetic nervous system and other vasoconstrictor hormones (Arnolda et al, Austr. NZ J. Med., 1999). The pacing model has been used previously in studies of the effects of both ANP and BNP on heart failure (Luchner et al, 2000; and Yamamoto et al, Am J Physiol, 1997).

In Vivo Results

Tables 13 and 14 show in vivo results in SHR rats of 7 week old and in Winstar-Kyoto rats of 7 week old respectively. The increase of urine secretion and the increase of cGMP expression have been measured 24 and 48 hours after injection of compound of Example 3. Concentrations of 1, 2 and 4 mg of compounds per kg of rats have been tested in comparison with saline solution. Control values have been taken before injection (pre-dose). The urine secretion (Vol.) is expressed in mL/day of urine exceeding the value at pre-dose. The cGMP expression (cGMP) is reported in ηmol/day and was measured by RIA method.

TABLE 13

| | Saline Solution | | 1 mg/kg | | 2 mg/kg | | 4 mg/kg | |
|---|---|---|---|---|---|---|---|---|
| Time Point | Vol. | cGMP | Vol. | cGMP | Vol. | cGMP | Vol. | cGMP |
| Pre-Dose | 0.0 ± 0.2 | 7.8 ± 1.5 | 0.0 ± 0.2 | 7.8 ± 1.5 | 0.0 ± 0.8 | 7.8 ± 1.5 | 0.0 ± 0.8 | 7.8 ± 1.5 |
| 24 h | 0.6 ± 0.2 | 19 ± 2 | −0.3 ± 0.3 | 29 ± 2 | 2.2 ± 0.7 | 35 ± 3 | 2.3 ± 0.8 | 40 ± 5 |
| 48 h | 2.6 ± 0.4 | 10.0 ± 0.4 | 3.1 ± 0.7 | 19 ± 1 | 4.2 ± 1.2 | 19 ± 4 | 2.0 ± 0.7 | 24 ± 3 |

TABLE 14

| | Saline Solution | | 1 mg/kg | | 2 mg/kg | | 4 mg/kg | |
|---|---|---|---|---|---|---|---|---|
| Time Point | Vol. | cGMP | Vol. | cGMP | Vol. | cGMP | Vol. | cGMP |
| Pre-Dose | 0.0 ± 0.9 | 19 ± 4 | 0.0 ± 0.4 | 19 ± 4 | 0.0 ± 0.4 | 19 ± 4 | 0.0 ± 0.4 | 19 ± 4 |
| 24 h | 1.0 ± 0.9 | 30 ± 5 | 1.5 ± 0.7 | 38 ± 3 | 8.9 ± 1.0 | 53 ± 3 | 2.3 ± 0.8 | 71 ± 5 |
| 48 h | 8.4 ± 2.2 | 24 ± 3 | 6.2 ± 1.5 | 24 ± 3 | 8.5 ± 0.6 | 22 ± 2 | 9.6 ± 1.0 | 36 ± 5 |

While the invention has been described in connection with specific embodiments thereof, it will be understood that it is capable of further modifications, and this application is intended to cover any variations, uses or adaptations of the invention following, in general, the principles of the invention, and including such departures from the present description as come within known or customary practice within the art to which the invention pertains, and as may be applied to the essential features hereinbefore set forth, and as follows in the scope of the appended claims.

1-26. (canceled)

27. A natriuretic peptide derivative comprising a NP peptide and a reactive entity coupled to the NP peptide, the reactive entity being capable of covalently bonding with a functionality on a blood component; wherein the NP peptide has a sequence of formula: 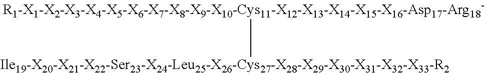
R₁-X₁-X₂-X₃-X₄-X₅-X₆-X₇-X₈-X₉-X₁₀-Cys₁₁-X₁₂-X₁₃-X₁₄-X₁₅-X₁₆-Asp₁₇-Arg₁₈-Ile₁₉-X₂₀-X₂₁-X₂₂-Ser₂₃-X₂₄-Leu₂₅-X₂₆-Cys₂₇-X₂₈-X₂₉-X₃₀-X₃₁-X₃₂-X₃₃-R₂
wherein
X₁ is Thr or absent;
X₂ is Ser, Thr, Ala or absent;
X₃ is Pro, Hpr, Val, or absent;
X₄ is Lys, D-Lys, Arg, D-Arg, Asn, Gln or absent;
X₅ is Met, Leu, Ile, an oxidatively stable Met-replacement amino acid, Ser, Thr or absent;
X₆ is Val, Ile, Leu, Met, Phe, Ala, D-Ala, Nle or absent;
X₇ is Gln, Asn, Arg, D-Arg, Asp, Lys, D-Lys or absent;
X₈ is Gly, Pro, Ala, D-Ala, Arg, D-Arg, Asp, Lys, D-Lys, Gln, Asn or absent;
X₉ is Ser, Thr or absent;
X₁₀ is Gly, Pro, Ala, D-Ala, Ser, Thr or absent;
X₁₂ is Phe, Tyr, Leu, Val, Ile, Ala, D-Ala, Phe with an isosteric replacement of its amide bond selected from the group consisting of N-α-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Arg, Lys, D-Lys, Asp, Gly, Ala, D-Ala or Pro;
X₁₅ is Lys, D-Lys, Arg, D-Arg, Asn, Gln or Asp;
X₁₆ is Met, Leu, Ile or an oxidatively stable Met-replacement amino acid;
X₂₀ is Ser, Gly, Ala, D-Ala or Pro;
X₂₁ is Ser, Gly, Ala, D-Ala, Pro, Val, Leu, or Ile;
X₂₂ is Ser, Gly, Ala, D-Ala, Pro, Gln or Asn;
X₂₄ is Gly, Ala, D-Ala or Pro;
X₂₆ is Gly, Ala, D-Ala or Pro;
X₂₈ is Lys, D-Lys, Arg, D-Arg, Asn, Gln, His or absent;
X₂₉ is Val, Ile, Leu, Met, Phe, Ala, D-Ala, Nle, Ser, Thr or absent;
X₃₀ is Leu, Nle, Ile, Val, Met, Ala, D-Ala, Phe, Tyr or absent;
X₃₁ is Arg, D-Arg, Asp, Lys, D-Lys or absent;
X₃₂ is Arg, D-Arg, Asp, Lys, D-Lys, Tyr, Phe, Trp, Thr, Ser or absent;
X₃₃ is His, Asn, Gln, Lys, D-Lys, Arg, D-Arg or absent;
R₁ is NH₂ or a N-terminal blocking group;
R₂ is COOH, CONH₂ or a C-terminal blocking group;
where a peptidic bond links Arg₁₈ and Ile₁₉ and the line between Cys₁₁ and Cys₂₇ represents a direct disulfide bridge.

28. The derivative defined in claim 27 wherein:
X₁ is Thr or absent;
X₂ is Ala or absent;
X₃ is Pro or absent;
X₄ is Arg or absent;
X₅ is Ser, Thr or absent;
X₆ is Leu, Ile, Nle, Met, Val, Ala, Phe or absent;
X₇ is Arg, D-Arg, Asp, Lys, D-Lys, Gln, Asn or absent;
X₈ is Arg, D-Arg, Asp, Lys, D-Lys, Gln, Asn or absent;
X₉ is Ser, Thr or absent;
X₁₀ is Ser, Thr or absent;
X₁₂ is Phe, Tyr, Leu, Val, Ile, Ala, D-Ala, Phe with an isosteric replacement of its amide bond selected from the group consisting of N-α-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Gly, Ala, D-Ala or Pro;
X₁₅ is Arg, Lys, D-Lys, or Asp;
X₁₆ is Met, Leu, Ile or an oxidatively stable Met-replacement amino acid;
X₂₀ is Gly, Ala, D-Ala or Pro;
X₂₁ is Ala, D-Ala, Val, Leu, or Ile;
X₂₂ is Gln or Asn;
X₂₄ is Gly, Ala, D-Ala or Pro;
X₂₆ is Gly, Ala, D-Ala or Pro;
X₂₈ is Asn, Gln, His, Lys, D-Lys, Arg, D-Arg or absent;
X₂₉ is Ser, Thr or absent;
X₃₀ is Phe, Tyr, Leu, Val, Ile, Ala or absent;
X₃₁ is Arg, D-Arg, Asp, Lys, D-Lys or absent;
X₃₂ is Tyr, Phe, Trp, Thr, Ser or absent;
X₃₃ is absent;
R₁ is NH₂ or a N-terminal blocking group;
R₂ is COOH, CONH₂ or a C-terminal blocking group.

29. The derivative of claim 28 wherein
X₁ is Thr or absent;
X₂ is Ala or absent;
X₃ is Pro or absent;
X₄ is Arg or absent;
X₅ is Ser or absent;
X₆ is Leu or absent;
X₇ is Arg, Asp or absent;
X₈ is Arg, Asp or absent;
X₉ is Ser or absent;
X₁₀ is Ser or absent;
X₁₂ is Phe or Phe with an isosteric replacement of its amide bond selected from the group consisting of N-α-methyl, methyl amino, hydroxylethyl, hydrazino, ethylene, sulfonamide and N-alkyl-β-aminopropionic acid;
X₁₃ is Gly;
X₁₄ is Gly;
X₁₅ is Arg or Asp;
X₁₆ is Met or Ile;
X₂₀ is Gly;
X₂₁ is Ala;
X₂₂ is Gln;
X₂₄ is Gly;
X₂₆ is Gly;
X₂₈ is Asn or absent;
X₂₉ is Ser or absent;
X₃₀ is Phe or absent;
X₃₁ is Arg, Asp or absent;
X₃₂ is Tyr or absent;
X₃₃ is absent;
R₁ is NH₂ or a N-terminal blocking group;
R₂ is COOH, CONH₂ or a C-terminal blocking group.

30. The derivative of claim 29, wherein the NP peptide is selected from the group consisting of SEQ ID NO: 1, SEQ ID NO: 8, SEQ ID NO: 12, SEQ ID NO: 13, SEQ ID NO: 15, SEQ ID NO: 17 and SEQ ID NO: 19.

31. The derivative of claim 29, selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 9, SEQ ID NO: 10, SEQ ID NO:11, SEQ ID NO: 14, SEQ ID NO: 16, SEQ ID NO:18 and SEQ ID NO: 20.

32. The derivative defined in claim 27, wherein:
X₁ is absent;
X₂ is Ser, Thr or absent;
X₃ is Pro, Hpr, Val or absent;
X₄ is Lys, D-Lys, Arg, D-Arg, Asn, Gln or absent;
X₅ is Met, Leu, Ile, an oxidatively stable Met-replacement amino acid or absent;
X₆ is Val, Ile, Leu, Met, Phe, Ala, D-Ala, Nle or absent;
X₇ is Gln, Asn or absent;
X₈ is Gly, Pro, Ala, D-Ala or absent;
X₉ is Ser, Thr or absent;
X₁₀ is Gly, Pro, Ala, D-Ala or absent;
X₁₂ is Phe, Tyr, Leu, Val, Ile, Ala, D-Ala, Phe with an isosteric replacement of its amide bond selected from the group consisting of N-α-methyl, methyl amino, hydroxylethyl, hydrazino, ethylene, sulfonamide and N-alkyl-β-aminopropionic acid, or a Phe-replacement amino acid conferring on said analog resistance to NEP enzyme;
X₁₃ is Gly, Ala, D-Ala or Pro;
X₁₄ is Arg, Lys, D-Lys, or Asp;
X₁₅ is Lys, D-Lys, Arg, D-Arg, Asn or Gln;
X₁₆ is Met, Leu, Ile or an oxidatively stable Met-replacement amino acid;
X₂₀ is Ser, Gly, Ala, D-Ala or Pro;
X₂₁ is Ser, Gly, Ala, D-Ala or Pro;
X₂₂ is Ser, Gly, Ala, D-Ala or Pro;
X₂₄ is Gly, Ala, D-Ala or Pro;
X₂₆ is Gly, Ala, D-Ala or Pro;
X₂₈ is Lys, D-Lys, Arg, D-Arg, Asn, Gln or absent;
X₂₉ is Val, Ile, Leu, Met, Phe, Ala, D-Ala, Nle or absent;
X₃₀ is Leu, Nle, Ile, Val, Met, Ala, D-Ala, Phe or absent;
X₃₁ is Arg, D-Arg, Asp, Lys, D-Lys or absent;
X₃₂ is Arg, D-Arg, Asp, Lys, D-Lys or absent;
X₃₃ is His, Asn, Gln, Lys, D-Lys, Arg, D-Arg or absent;
R₁ is NH₂ or a N-terminal blocking group;
R₂ is COOH, CONH₂ or a C-terminal blocking group.

33. The derivative of claim 32 wherein:
X₁ is absent;
X₂ is Ser or absent;
X₃ is Pro or absent;
X₄ is Lys or absent;
X₅ is Met, Ile or absent;
X₆ is Val or absent;
X₇ is Gln or absent;
X₈ is Gly or absent;
X₉ is Ser or absent;
X₁₀ is Gly or absent;
X₁₂ is Phe or Phe with an isosteric replacement of its amide bond selected from the group consisting of N-α-methyl, methyl amino, hydroxylethyl, hydrazino, ethylene, sulfonamide and N-alkyl-β-aminopropionic acid;
X₁₃ is Gly;
X₁₄ is Arg or Asp;
X₁₅ is Lys or Arg;
X₁₆ is Met or Ile;
X₂₀ is Ser;
X₂₁ is Ser;
X₂₂ is Ser;
X₂₄ is Gly;
X₂₆ is Gly;
X₂₈ is Lys, Arg or absent;
X₂₉ is Val or absent;
X₃₀ is Leu or absent;
X₃₁ is Arg, Asp or absent;
X₃₂ is Arg, Asp or absent;
X₃₃ is His or absent.

34. The derivative of claim 33 wherein the NP peptide is selected from the group consisting of SEQ ID NO: 21, SEQ ID NO: 22, SEQ ID NO: 23, SEQ ID NO: 25, SEQ ID NO: 28, SEQ ID NO: 31, SEQ ID NO: 34, SEQ ID NO: 37, SEQ ID NO: 39, SEQ ID NO: 42, SEQ ID NO: 45, SEQ ID NO: 48 and SEQ ID NO: 51.

35. The derivative of claim 33 selected from the group consisting of SEQ ID NO: 24, SEQ ID NO: 26, SEQ ID NO: 27, SEQ ID NO: 29, SEQ ID NO: 30, SEQ ID NO: 32, SEQ ID NO: 33, SEQ ID NO: 35, SEQ ID NO: 36, SEQ ID NO: 38, SEQ ID NO: 40, SEQ ID NO: 41, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 54, SEQ ID NO: 55, SEQ ID NO: 56 and SEQ ID NO: 57.

36. The derivative of claim 27, being capable of selectively covalently bonding with a single functionality on the blood component whith a degree of selectivity of 80% or more.

37. The derivative of claim 36, wherein the derivative bonds the blood component in a ratio 1:1 derivative:blood component.

38. The derivative of claim 27, wherein the reactive entity is a maleimide or a maleimido-containing group.

39. The derivative of claim 38, wherein the reactive entity is MPA.

40. A pharmaceutical composition comprising the derivative of claim 27 in combination with a pharmaceutically acceptable carrier.
